(19) United States
(12) Patent Application Publication
(10) Pub. No.: US 2025/0257393 A1
(43) Pub. Date: Aug. 14, 2025

(54) METHODS AND COMPOSITIONS FOR DETECTING NUCLEIC ACIDS IN A FIXED BIOLOGICAL SAMPLE

(71) Applicant: 10x Genomics, Inc., Pleasanton, CA (US)

(72) Inventors: Layla Katiraee, Castro Valley, CA (US); Jennifer Chew, Hayward, CA (US)

(73) Assignee: 10x Genomics, Inc., Pleasanton, CA (US)

(21) Appl. No.: 18/640,819

(22) Filed: Apr. 19, 2024

Related U.S. Application Data
(60) Provisional application No. 63/497,635, filed on Apr. 21, 2023.

(51) Int. Cl. C12Q 1/6874; C12N 15/10; C12Q 1/6806; C12Q 1/6853; G01N 1/30

(52) U.S. Cl. CPC C12Q 1/6874; C12N 15/1096; C12Q 1/6806; C12Q 1/6853; G01N 1/30; C12Q 2600/156; G01N 2001/305

(57) ABSTRACT

Provided herein are method for determining a location of nucleic acids in fixed biological samples, in which the method includes use of a template switching oligonucleotide (TSO) and a randomer during second strand synthesis.

### CROSS-REFERENCE TO RELATED APPLICATION

This application claims priority to U.S. Provisional Patent Application No. 63/497,635, filed Apr. 21, 2023, the content of which is incorporated herein by reference in its entirety.

### BACKGROUND

Cells within a tissue of a subject have differences in cell morphology and/or function due to varied analyte levels (e.g., gene and/or protein expression) within the different cells. The specific position of a cell within a tissue (e.g., the cell's position relative to neighboring cells or the cell's position relative to the tissue microenvironment) can affect, e.g., the cell's morphology, differentiation, fate, viability, proliferation, behavior, signaling, and cross-talk with other cells in the tissue.

Spatial heterogeneity has been previously studied using techniques that provide data for a handful of analytes in the context of an intact tissue or a portion of a tissue, or provides substantial analyte data for dissociated tissue (i.e., single cells), but fail to provide information regarding the position of a single cell in a parent biological sample (e.g., tissue sample). Techniques for spatial analysis have advanced but can still be affected by use of samples that may be degraded (e.g., as in a formalin-fixed, paraffin embedded (FFPE) sample), in which analytes within such samples may be difficult to interrogate.

### SUMMARY

The present disclosure provides methods for determining the location of nucleic acids in a fixed biological sample, such as a fixed tissue sample, in which previous knowledge of the sequence of the nucleic acid is not known. Spatial transcriptomics is the evaluation of gene expression, up to and including the whole transcriptome, at a spatial level in a biological sample. For example, nucleic acids such as mRNA transcripts, such as mammalian mRNA transcripts, can be captured using a common transcript sequence, such as a poly(A) mRNA tail. The methods for direct capture of mRNA from a biological sample presume that the target nucleic acids such as mRNA are not, or are minimally, degraded. As such, fresh sample or fresh frozen samples have historically been used in direct mRNA capture methods for spatial transcriptomics. However, fixed biological samples such as pathological biopsy samples are not fresh, but are instead fixed for evaluation and storage, such as formalin fixed paraffin embedded tissue samples. Additionally, fresh and/or fresh frozen tissues can be fixed as well in methanol, acetone, formalin, formaldehyde, etc. for preservation of the tissue. Fixing of tissue samples and other biological samples can affect the availability and integrity of the nucleic acids, such that mRNA in fixed biological samples is oftentimes degraded and difficult to capture and assay.

The method disclosed herein provides a solution to increase the usability of degraded mRNA from fixed biological samples. The methods disclosed herein employ at least two different primers, in which a first primer includes a sequence of a template switching oligonucleotide (TSO) and a second primer is a randomer sequence. Without wishing to be limited by mechanism or theory, the use of at least two different primers during second strand synthesis may enhance and increase detection of captured nucleic acids from fixed biological samples. In some non-limiting embodiments, such methods can be used to interrogate nucleic acids of fixed samples, e.g., FFPE samples, where the nucleic acids may be compromised in regard to quality to a greater degree than a non-fixed sample.

Accordingly, in one non-limiting aspect, the present disclosure features a method of generating a plurality of second strand synthesis products from a plurality of nucleic acids in a fixed biological sample for spatial transcriptomics, the method comprising: (a) contacting a fixed biological sample with a substrate, wherein the substrate comprises a plurality of capture probes; (b) hybridizing the plurality of nucleic acids to the plurality of capture probes; (c) generating a population of extended capture probes by reverse transcription; and (d) performing second strand synthesis on the population of extended capture probes in the presence of a plurality of first primers and a plurality of second primers.

In some instances, the method thereby generates a plurality of second strand synthesis products from a plurality of nucleic acids in a fixed biological sample for spatial transcriptomics.

In some instances, the capture probe of the plurality of capture probes comprises a spatial barcode and a capture domain.

In some instances, the population of extended capture probes includes: (i) a first population of extended capture probes comprising a cDNA sequence of the hybridized nucleic acid and a template switching oligonucleotide (TSO) sequence, or a complement thereof; and (ii) a second population of extended capture probes comprising a cDNA sequence of the nucleic acid and lacking a TSO sequence, or a complement thereof.

In some instances, the first primer is a TSO primer configured to hybridize to all or a portion of the TSO sequence of an extended capture probe. In some instances, the first primer can include a sequence that is complementary to the TSO sequence of an extended capture probe.

In some instances, the first primer lacks a poly(G) sequence.

In some instances, the second primer is a randomer primer or includes randomers. In some instances, the second primer can include a sequence of a randomer.

In some instances, the second primer comprises a defined sequence at the 5′ end of the randomer. The defined sequence can include one or more of a universal sequence (e.g., a primer sequence or a sequence compatible with a sequencing platform, or both) and a cleavage sequence.

In some instances, the method further includes: determining (i) all of the sequence of the spatial barcode of the extended capture probes and (ii) the sequence of all or a portion of the nucleic acid, and using (i) and (ii) to identify the location of the nucleic acid in the fixed biological sample.

In some instances, the reverse transcription comprises: (i) extending the capture probe using the hybridized nucleic acid as a template, and appending a polynucleotide sequence to the end of the extended capture probe; (ii) hybridizing a TSO comprising a polyribonucleotide sequence complementary to the polynucleotide sequence, and extending further the extended capture probe to include the complementary sequences of the TSO, thereby generating the first population of extended capture probes comprising the cDNA sequences and the complementary TSO sequences; and (iii) extending the capture probes using the hybridized nucleic acids as a template, thereby generating the second population of extended capture probes comprising the cDNA sequences. Operations (i) and (iii) can occur in any order or simultaneously.

In some instance, the second strand synthesis comprises: hybridizing the complementary TSO primers to the TSO sequence on the cDNA of the first population of extended capture probes and extending the TSO primer using the cDNA as a template, thereby generating second strand synthesis products comprising the cDNA sequences, or complements thereof, and the capture probe sequences, or complements thereof.

In some instances, the second strand synthesis comprises: hybridizing the randomers to one or both of the first population of extended capture probes and the second population of extended capture probes (e.g., at a random position) and extending the randomers using the cDNA as a template, thereby generating second strand synthesis products comprising the cDNA sequences, or complements thereof, and the capture probe sequences, or complements thereof. In another aspect, the present disclosure encompasses a method of processing an analyte (e.g., a nucleic acid) from a biological sample, comprising: (a) hybridizing the analyte from the biological sample to a capture probe, wherein the capture probe comprises a capture domain and a spatial barcode; (b) performing reverse transcription using the hybridized nucleic acid as a template in the presence of a template switching oligonucleotide (TSO), thereby generating a population of complementary DNA (cDNA) molecules of the analyte, wherein a first cDNA molecule in the population comprises a reverse complement of a template switching oligonucleotide (rcTSO) and wherein a second cDNA molecule in the population lacks a rcTSO; and (c) performing second strand synthesis in the presence of a first primer and a second primer, wherein the first primer comprises a sequence that hybridizes to the rcTSO sequence, if it is present, and wherein the second primer is a randomer.

In some instances, the reverse transcription comprises: (i) providing the TSO in proximity to a 5′ end of the analyte; and (ii) extending the capture probe using the TSO and the analyte as a template, thereby generating the first cDNA molecule comprising the rcTSO and a sequence that is complementary to all or a portion of the analyte. In other instances, the reverse transcription comprises: (i) extending the capture probe using the analyte as a template, wherein the template lacks a TSO, thereby generating the second cDNA molecule comprising a sequence that is complementary to all or a portion of the analyte.

In some instances, the second strand synthesis comprises: (i) hybridizing the TSO sequence of the first primer to the rcTSO of the first cDNA molecule; and (ii) extending the TSO sequence of the first primer using the extended capture probe as a template, thereby generating a second strand, wherein the second strand is complementary to all or a portion of the analyte and all or a portion of the capture probe. In other instances, the second strand synthesis comprises: (i) hybridizing the randomer to the first cDNA molecule or the second cDNA molecule (e.g., at a random position); and (ii) extending the randomer using the extended capture probe as a template, thereby generating a second strand, wherein the second strand is complementary to all or a portion of the analyte and all or a portion of the capture probe.

In yet another non-limiting aspect, the present disclosure features a kit. In some instances, the kit comprises: (a) a substrate comprises a plurality of captures probes attached to the surface of the substrate, wherein a capture probe of the plurality of capture probes comprises a spatial barcode and a capture domain; (b) one or more reagents selected from a buffer, a plurality of dNTPs, a plurality of template switching oligonucleotides (TSOs), a plurality of sequences complementary to the TSOs or complementary to a portion of the TSOs, and a plurality of randomer sequences; and (c) one or more enzymes selected from a reverse transcriptase and a polymerase. In some instances, the kit further includes: (d) instructions for performing any method described herein.

In any embodiments herein, the capture probe can further include one or more functional domains, a unique molecular identifier (UMI), a cleavage domain, and combinations thereof.

In any embodiments herein, the reverse transcription is conducted in the presence of a reverse transcription enzyme comprising one or more of terminal transferase activity, template switching ability, strand displacement ability, or combinations thereof. In some instances, the reverse transcription enzyme comprises a Moloney Murine Leukemia Virus (M-MLV) reverse transcriptase enzyme (e.g., M-MLV reverse transcriptase enzyme 42B).

In any embodiments herein, the TSO is about 10 to 50 nucleotides in length. In some instances, the TSO comprises DNA, RNA, or a combination of any of these. In some instances, the TSO comprises a homopolymer guanine sequence (e.g., poly(G), which can include G as a DNA base or an RNA base) that hybridizes to a homopolymer cytosine sequence (e.g., poly(C)) on the capture probe or the extended capture probe. In some instances, the TSO comprises a sequence that hybridizes to the capture probe or the extended capture probe.

In any embodiments herein, the first primer includes a sequence for the TSO or a sequence for a portion of the TSO. In some instances, the first primer may lack a poly(G) sequence.

In any embodiments herein, the rcTSO comprises DNA.

In any embodiments herein, the randomer is about 4 to 16 nucleotides in length. In some instances, the randomer is a random hexamer.

In any embodiments herein, second strand synthesis is performed concurrent with or immediately after first strand cDNA synthesis.

In any embodiments herein, the method further includes releasing the second strand synthesis products. In some instances, releasing the second strand synthesis products comprises physical denaturation, enzymatic reaction, or chemical denaturation.

In any embodiments herein, the method further includes (e.g., prior to hybridizing the analyte to the capture probe): permeabilizing the biological sample with a permeabilization agent. In some instances, the permeabilization agent is selected from an organic solvent, a detergent, and an enzyme, or a combination thereof. In other instances, the permeabilization agent is selected from an endopeptidase (e.g., pepsin or proteinase K), a protease (e.g., proteinase K), urea, sodium dodecyl sulfate (SDS), polyethylene glycol (PEG), polyethylene glycol tert-octylphenyl ether, polysorbate 80, and polysorbate 20, N-lauroylsarcosine sodium salt solution, saponin, Triton X-100™, and Tween-20™.

In any embodiments herein, the method further includes (e.g., prior to contacting the biological sample with the substrate): fixing the biological sample. In some instances, the step of fixing the biological sample is performed using one or both of methanol and acetone.

In any embodiment herein, the fixed biological sample is fixed with methanol, acetone, paraformaldehyde (PFA), formaldehyde, another fixative (e.g., any described herein), or a combination thereof.

In any embodiments herein, the biological sample comprises a tissue section. In some instances, the biological sample comprises a formalin-fixed, paraffin-embedded (FFPE) sample, a frozen sample, or a fresh sample. In some instances, the fixed biological sample comprises a formalin-fixed, paraffin-embedded (FFPE) sample. In some instances, the FFPE tissue sample is deparaffinized. In some instances, the FFPE tissue sample is decrosslinked with a decrosslinking agent (e.g., citrate or any described herein).

In any embodiment herein, the biological sample is treated with an RNase inactivating agent (e.g., an RNase inhibitor, ribonucleoside vanadyl complex, EDTA, etc.).

In any embodiment herein, the biological sample (e.g., fixed biological sample) is removed from the substrate after hybridization of the analyte (e.g., nucleic acid) to the capture probe.

In any embodiment herein, the biological sample (e.g., fixed biological sample) was previously stained. In some instances, the biological sample (e.g., fixed biological sample) was previously stained using hematoxylin and eosin, immunofluorescence, or immunohistochemistry. In some instances, the biological sample was previously stained using hematoxylin and/or eosin.

In any embodiment herein, the analyte comprises a nucleic acid. In some instances, the nucleic acid is an RNA molecule (e.g., an mRNA molecule). In some instances, the nucleic acid comprises a single nucleotide polymorphism (SNP). In some instances, the nucleic acid is of non-human origin or non-mouse origin.

In any embodiment herein, the method further comprises: determining (i) all of the sequence of the spatial barcode of the extended capture probes and (ii) the sequence of all or a portion of the nucleic acid, and using (i) and (ii) to identify the location of the nucleic acid in the fixed biological sample.

In any embodiment herein, a determining step comprises amplifying all or part of the analyte, thereby producing an amplifying product. In some instances, the amplified product comprises (i) all or part of the analyte, or a complement thereof, and (ii) all or a part of the spatial barcode, or a complement thereof.

In any embodiment herein, a determining step comprises sequencing. In some instances, sequencing comprises in situ sequencing, Sanger sequencing methods, next-generation sequencing methods, or nanopore sequencing.

All publications, patents, and patent applications mentioned in this specification are herein incorporated by reference to the same extent as if each individual publication, patent, patent application, or item of information was specifically and individually indicated to be incorporated by reference. To the extent publications, patents, patent applications, and items of information incorporated by reference contradict the disclosure contained in the specification, the specification is intended to supersede and/or take precedence over any such contradictory material.

Where values are described in terms of ranges, it should be understood that the description includes the disclosure of all possible sub-ranges within such ranges, as well as specific numerical values that fall within such ranges irrespective of whether a specific numerical value or specific sub-range is expressly stated.

The term “each,” when used in reference to a collection of items, is intended to identify an individual item in the collection but does not necessarily refer to every item in the collection, unless expressly stated otherwise, or unless the context of the usage clearly indicates otherwise. Various embodiments of the features of this disclosure are described herein. However, it should be understood that such embodiments are provided merely by way of example, and numerous variations, changes, and substitutions can occur to those skilled in the art without departing from the scope of this disclosure. It should also be understood that various alternatives to the specific embodiments described herein are also within the scope of this disclosure.

### DESCRIPTION OF DRAWINGS

The following drawings illustrate certain embodiments of the features and advantages of this disclosure. These embodiments are not intended to limit the scope of the appended claims in any manner. Like reference symbols in the drawings indicate like elements.

FIG. 1A shows an exemplary sandwiching process where a first substrate (e.g., a slide), including a biological sample, and a second substrate (e.g., array slide) are brought into proximity with one another.

FIG. 1B shows a fully formed sandwich configuration creating a chamber formed from the one or more spacers, the first substrate, and the second substrate.

FIG. 2A shows a perspective view of an exemplary sample handling apparatus in a closed position.

FIG. 2B shows a perspective view of an exemplary sample handling apparatus in an open position.

FIG. 3A shows the first substrate angled over (superior to) the second substrate.

FIG. 3B shows that as the first substrate lowers, and/or as the second substrate rises, the dropped side of the first substrate may contact a drop of reagent medium.

FIG. 3C shows a full closure between the first substrate and the second substrate with one or more spacers contacting both the first substrate and the second substrate.

FIG. 4A shows a side view of the angled closure workflow.

FIG. 4B shows a top view of the angled closure workflow.

FIG. 5 is a schematic diagram showing an example of a barcoded capture probe as described herein.

FIG. 6 shows a schematic illustrating a cleavable capture probe.

FIG. 7 shows exemplary capture domains on capture probes.

FIG. 8 shows an exemplary arrangement of barcoded features within an array.

FIG. 9A shows an exemplary workflow for performing templated capture and producing a ligation product, and FIG. 9B shows an exemplary workflow for capturing a ligation product from FIG. 9A on a substrate.

FIG. 10 shows an exemplary schematic illustrating a barcoded capture probe, the capture of a polyadenylated mRNA from, for example, a biological sample, and an exemplary operation for generating a final molecule used for analyzing the originally captured mRNA.

FIG. 11 shows two exemplary scenarios for performing second strand synthesis in the presence of a non-limiting primer in scenario 1 (e.g., a random hexamer primer) and a non-limiting additional primer in scenario 2 (e.g., a TSO primer).

FIG. 12 shows an exemplary experimental design configuration for comparing two spatial chemistries on a variety of different FFPE human tissue samples.

FIG. 13 shows an exemplary workflow for capturing nucleic acids and performing second strand synthesis in the presence of a template switching oligonucleotide (TSO) or a randomer, as depicted in FIG. 11.

FIG. 14 shows exemplary tissue images reporting UMIs counts (log 10) when comparing the two spatial chemistries on various FFPE tissue samples.

FIG. 15 shows exemplary gene clustering data when comparing the two spatial chemistries on various FFPE tissue samples from FIG. 12.

FIG. 16 shows exemplary raw data related to library quality for two different spatial chemistries performed on different FFPE tissue sample types from FIG. 12, using genome reference consortium human build 38 (GRCh38).

FIG. 17 shows exemplary raw data related to sensitivity when comparing two different spatial chemistries on various FFPE tissue sample types from FIG. 12, using GRCh38.

FIG. 18 and FIG. 19 shows graphs providing exemplary saturation curves when comparing two different spatial chemistries on various FFPE tissue sample types from FIG. 12. A=mRNA capture and combination primers from FFPE sample, B=RTL capture from FFPE sample, C=replicate for RTL capture from FFPE sample.

FIG. 20 shows exemplary gene expression analysis results for four genes when comparing two different spatial chemistries on FFPE human tonsil tissue samples.

### DETAILED DESCRIPTION

Described herein are methods and compositions that can be used in analyzing nucleic acids from fixed biological samples, such as formalin fixed paraffin embedded samples, samples where the nucleic acids are modified by the fixation method, thereby providing challenges in their evaluation in downstream assays. As demonstrated herein, nucleic acids such as mRNA from FFPE tissue samples can be captured and, surprisingly, assayed using a combination of TSO based methods in combination with randomer second strand synthesis methods, thereby providing additional avenues of studying gene expression in tissue samples that have been previously fixed, wherein the nucleic acid quality is questionable.

Methods and compositions for spatially identifying and correlating the location of nucleic acids within a biological sample have been known to follow at least two paths: 1) capturing nucleic acids from a sample onto a spatially arrayed slide, or 2) performing in situ analysis of nucleic acids. These spatial transcriptomics methods relate to a new and greatly expanding technology area that provides data about locations of RNA, DNA, etc. within a cell or tissue. This explosion of new information opens the door to discoveries which were not previously available to scientists.

While the world of spatial transcriptomics is exploding, methods and compositions for performing for performing spatial transcriptomics on different sample types are still needed. For example, some methods for practicing spatial transcriptomics rely on the direct capture of a nucleic acid such as mRNA from a tissue sample onto a slide. The directly captured mRNA can then be transcribed, second strand synthesis performed, and the cDNA products released, sequenced, and correlated back to the original tissue from whence they came. However, direct capture of mRNA from a tissue sample is typically used when tissues are fresh or fresh frozen tissue samples, as the quality of mRNA in these tissue types is normally high (i.e., mRNA is not so degraded). Conversely, nucleic acids such as mRNA found in a fixed tissue sample, for example a FFPE tissue sample, are typically not of high quality due to the fixing process, as such mRNA can be degraded to different degrees in different tissue types, or within a tissue sample. Different methods have been developed, such as the RNA templated ligation (RTL) method described herein, which is an indirect measure of the presence of a nucleic acid at a location in a tissue sample. The RTL method takes into account the nature of degraded mRNA which is typically present in an FFPE tissue sample, and provides probes that are sequence specific to the target nucleic acid, thereby providing a proxy that indirectly identifies the presence and location of a nucleic acid in a fixed biological sample.

The present disclosure reports a method where direct capture of nucleic acids such as mRNA from a fixed or FFPE tissue sample is performed, and the ability to analyze the captured nucleic acids is increased or enhanced by combining two second strand synthesis primer scenarios, thereby generating an increased amount of target nucleic acid product to be analyzed. The disclosed combination second strand synthesis method, surprisingly, can be used to analyze mRNA from a fixed tissue sample, even if that mRNA may be degraded, and assayed to generate spatial transcriptomics data that is comparable to data generated using the RTL method that was specifically developed to target degraded nucleic acid analysis from fixed biological samples.

As disclosed here, the use of two types of primers can enhance second strand synthesis when performing direct capture of nucleic acids, for example mRNA, from a FFPE tissue sample. For example and without limitation, a template switching oligonucleotide (TSO) can provide a sequence for use during second strand synthesis of a first strand cDNA product derived from a captured mRNA. In use, template switching can include appending a TSO to a captured nucleic acid, such that the nucleic acid-TSO complex is used as a template for second strand synthesis. Yet, a degraded nucleic acid, such as degraded mRNA from a FFPE tissue sample, may be less amenable for template switching, such that addition of the TSO to a captured nucleic acid or formation of the nucleic acid-TSO complex may be limited or absent. As described herein, second strand synthesis can be rescued by incorporating one or more additional, alternative primer(s) (e.g., in addition to a primer having a sequence that is complementary to the TSO sequence) into the workflow for second strand synthesis of nucleic acids on a spatial array. In non-limiting embodiments, the one or more alternative primers comprise random sequences, in effect a set of randomer primers, which can hybridize to one or more sequences within the first strand synthesis product. Such a combination approach can be used with any methods described herein to provide spatial analysis of nucleic acids from a fixed biological sample (e.g., a biological sample, such as a tissue section).

### I. Spatial Analysis

Spatial analysis methodologies and compositions described herein can provide a vast amount of analyte and/or expression data for a variety of analytes within a biological sample at high spatial resolution, while retaining native spatial context. Spatial analysis methods and compositions can include, e.g., the use of a capture probe including a spatial barcode (e.g., a nucleic acid sequence that provides information as to the location or position of an analyte within a cell or a tissue sample (e.g., mammalian cell or a mammalian tissue sample) and a capture domain that is capable of binding to an analyte (e.g., a protein and/or a nucleic acid) produced by and/or present in a cell. Spatial analysis methods and compositions can also include the use of a capture probe having a capture domain that captures an intermediate agent for indirect detection of an analyte. For example, the intermediate agent can include a nucleic acid sequence (e.g., a barcode) associated with the intermediate agent. Detection of the intermediate agent is therefore indicative of the analyte in the cell or tissue sample. Intermediate agents (e.g., ligation products or other sequences) can serve as proxies of target analytes in the methods and compositions herein.

Non-limiting aspects of spatial analysis methodologies and compositions are described in U.S. Pat. Nos. 11,447,807, 11,352,667, 11,168,350, 11,104,936, 11,008,608, 10,995,361, 10,913,975, 10,774,374, 10,724,078, 10,640,816, 10,494,662, 10,480,022, 10,364,457, 10,317,321, 10,059,990, 10,041,949, 10,030,261, 10,002,316, 9,879,313, 9,783,841, 9,727,810, 9,593,365, 8,951,726, 8,604,182, and 7,709,198; U.S. Patent Application Publication Nos. 2020/0239946, 2020/0080136, 2020/0277663, 2019/0330617, 2020/0256867, 2020/0224244, 2019/0085383, and 2013/0171621; PCT Publication Nos. WO2018/091676, WO2020/176788, WO2017/144338, and WO2016/057552; Non-patent literature references Rodriques et al., Science 363 (6434): 1463-1467, 2019; Lee et al., Nat. Protoc. 10 (3): 442-458, 2015; Trejo et al., PLOS ONE 14 (2): e0212031, 2019; Chen et al., Science 348 (6233): aaa6090, 2015; Gao et al., BMC Biol. 15:50, 2017; and Gupta et al., Nature Biotechnol. 36:1197-1202, 2018; the Visium Spatial Gene Expression Reagent Kits User Guide (e.g., Rev F, dated January 2022); and/or the Visium Spatial Gene Expression Reagent Kits-Tissue Optimization User Guide (e.g., Rev E, dated February 2022), both of which are available at the 10× Genomics Support Documentation website, and can be used herein in any combination, and each of which is incorporated herein by reference in their entireties. Further non-limiting aspects of spatial analysis methodologies and compositions are described herein.

Some general terminology that may be used in this disclosure can be found in Section (I)(b) of PCT Publication No. WO2020/176788 and/or U.S. Patent Application Publication No. 2020/0277663. Typically, a “barcode” is a label, or identifier, that conveys or is capable of conveying information (e.g., information about an analyte in a sample, a bead, and/or a capture probe). A barcode can be part of an analyte, or independent of an analyte. A barcode can be attached to an analyte. A particular barcode can be unique relative to other barcodes. For the purpose of this disclosure, an “analyte” can include any biological substance, structure, moiety, or component to be analyzed. The term “target” can similarly refer to an analyte of interest. Analytes can be broadly classified into one of two groups: nucleic acid analytes, and non-nucleic acid analytes. Examples of nucleic acid analytes include, but are not limited to, DNA, RNA (e.g., mRNA), and combinations thereof. Examples of non-nucleic acid analytes include, but are not limited to, lipids, carbohydrates, peptides, proteins, glycoproteins (N-linked or O-linked), lipoproteins, phosphoproteins, specific phosphorylated or acetylated variants of proteins, amidation variants of proteins, hydroxylation variants of proteins, methylation variants of proteins, ubiquitylation variants of proteins, sulfation variants of proteins, viral proteins (e.g., viral capsid, viral envelope, viral coat, viral accessory, viral glycoproteins, viral spike, etc.), extracellular and intracellular proteins, antibodies, and antigen binding fragments. In some embodiments, the analyte(s) can be localized to subcellular location(s), including, for example, organelles, e.g., mitochondria, Golgi apparatus, endoplasmic reticulum, chloroplasts, endocytic vesicles, exocytic vesicles, vacuoles, lysosomes, etc. In some embodiments, analyte(s) can be peptides or proteins, including without limitation antibodies and enzymes. Additional examples of analytes can be found in Section (I)(c) of PCT Publication No. WO2020/176788 and/or U.S. Patent Application Publication No. 2020/0277663. In some embodiments, an analyte can be detected indirectly, such as through detection of an intermediate agent, for example, a ligation product or an analyte capture agent (e.g., an oligonucleotide-conjugated antibody), such as those described herein.

A “biological sample” is typically obtained from the subject for analysis using any of a variety of techniques including, but not limited to, biopsy, surgery, and laser capture microscopy (LCM), and generally includes cells and/or other biological material from the subject. In some embodiments, a biological sample is a tissue sample. In some embodiments, the biological sample (e.g., tissue sample) is a tissue microarray (TMA). A tissue microarray contains multiple representative tissue samples-which can be from different tissues or organisms-assembled on a single histologic slide. The TMA can therefore allow for high throughput analysis of multiple specimens at the same time. Tissue microarrays are paraffin blocks produced by extracting cylindrical tissue cores from different paraffin donor blocks and re-embedding these into a single recipient (microarray) block at defined array coordinates.

The biological sample as used herein can be any suitable biological sample described herein or known in the art. In some embodiments, the biological sample is a tissue sample. In some embodiments, the tissue sample is a solid tissue sample. In some embodiments, the biological sample is a tissue section (e.g., a fixed tissue section). In some embodiments, the tissue is flash-frozen and sectioned. Any suitable method described herein or known in the art can be used to flash-freeze and section the tissue sample. In some embodiments, the biological sample, e.g., the tissue, is flash-frozen using liquid nitrogen before sectioning. In some embodiments, the biological sample, e.g., a tissue sample, is flash-frozen using nitrogen (e.g., liquid nitrogen), isopentane, or hexane.

In some embodiments, the biological sample, e.g., the tissue, is embedded in a matrix e.g., optimal cutting temperature (OCT) compound to facilitate sectioning. OCT compound is a formulation of clear, water-soluble glycols and resins, providing a solid matrix to encapsulate biological (e.g., tissue) specimens. In some embodiments, the sectioning is performed by cryosectioning, for example using a microtome. In some embodiments, the methods further comprise a thawing step, after the cryosectioning.

The biological sample can be from a mammal. In some instances, the biological sample is from a human, mouse, or rat. In addition to the subjects described above, the biological sample can be obtained from non-mammalian organisms (e.g., a plant, an insect, an arachnid, a nematode (e.g., Caenorhabditis elegans), a fungus, an amphibian, or a fish (e.g., zebrafish)). A biological sample can be obtained from a prokaryote such as a bacterium, e.g., Escherichia coli, Staphylococci or Mycoplasma pneumoniae; an archaea; a virus such as Hepatitis C virus or human immunodeficiency virus; or a viroid. A biological sample can be obtained from a eukaryote, such as a patient derived organoid (PDO) or patient derived xenograft (PDX). The biological sample can include organoids, a miniaturized and simplified version of an organ produced in vitro in three dimensions that shows realistic micro-anatomy. Organoids can be generated from one or more cells from a tissue, embryonic stem cells, and/or induced pluripotent stem cells, which can self-organize in three-dimensional culture owing to their self-renewal and differentiation capacities. In some embodiments, an organoid is a cerebral organoid, an intestinal organoid, a stomach organoid, a lingual organoid, a thyroid organoid, a thymic organoid, a testicular organoid, a hepatic organoid, a pancreatic organoid, an epithelial organoid, a lung organoid, a kidney organoid, a gastruloid, a cardiac organoid, or a retinal organoid. Subjects from which biological samples can be obtained can be healthy or asymptomatic individuals, individuals that have or are suspected of having a disease (e.g., cancer) or a pre-disposition to a disease, and/or individuals that are in need of therapy or suspected of needing therapy.

Biological samples can be derived from a homogeneous culture or population of the subjects or organisms mentioned herein or alternatively from a collection of several different organisms, for example, in a community or ecosystem.

Biological samples can include one or more diseased cells. A diseased cell can have altered metabolic properties, gene expression, protein expression, and/or morphologic features. Examples of diseases include inflammatory disorders, metabolic disorders, nervous system disorders, and cancer. Cancer cells can be derived from solid tumors, hematological malignancies, cell lines, or obtained as circulating tumor cells.

In some embodiments, the biological sample, e.g., the tissue sample, is fixed in a fixative including alcohol, for example methanol. In some embodiments, instead of methanol, acetone, or an acetone-methanol mixture can be used. In some embodiments, the fixation is performed after sectioning. In some instances, when the biological sample is fixed with a fixative including an alcohol (e.g., methanol or acetone-methanol mixture), it is not decrosslinked afterward. In some preferred embodiments, the biological sample is fixed with a fixative including an alcohol (e.g., methanol or an acetone-methanol mixture) after freezing and/or sectioning. In some instances, the biological sample is flash-frozen, and then the biological sample is sectioned and fixed (e.g., using methanol, acetone, or an acetone-methanol mixture). In some instances when methanol, acetone, or an acetone-methanol mixture is used to fix the biological sample, the sample is not decrosslinked at a later step. In instances when the biological sample is frozen (e.g., flash frozen using liquid nitrogen and embedded in OCT) followed by sectioning and alcohol (e.g., methanol, acetone-methanol) fixation or acetone fixation, the biological sample is referred to as “fresh frozen”. In some embodiments, fixation of the biological sample e.g., using acetone and/or alcohol (e.g., methanol, acetone-methanol) is performed while the sample is mounted on a substrate (e.g., glass slide, such as a positively charged glass slide).

In some embodiments, the biological sample, e.g., the tissue sample, is fixed e.g., immediately after being harvested from a subject. In such embodiments, the fixative is preferably an aldehyde fixative, such as paraformaldehyde (PFA) or formalin. In some embodiments, the fixative induces crosslinks within the biological sample. In some embodiments, after fixing e.g., by formalin or PFA, the biological sample is dehydrated via sucrose gradient. In some instances, the fixed biological sample is treated with a sucrose gradient and then embedded in a matrix e.g., OCT compound. In some instances, the fixed biological sample is not treated with a sucrose gradient, but rather is embedded in a matrix e.g., OCT compound after fixation. In some embodiments when a fixed frozen tissue sample is treated with a sucrose gradient, it can be rehydrated with an ethanol gradient. In some embodiments, the PFA or formalin fixed biological sample, which can be optionally dehydrated via sucrose gradient and/or embedded in OCT compound, is then frozen e.g., for storage or shipment. In such instances, the biological sample is referred to as “fixed frozen”. In preferred embodiments, a fixed frozen biological sample is not treated with methanol. In preferred embodiments, a fixed frozen biological sample is not paraffin embedded. Thus, in preferred embodiments, a fixed frozen biological sample is not deparaffinized. In some embodiments, a fixed frozen biological sample is rehydrated in an ethanol gradient.

In some instances, the biological sample (e.g., a fixed frozen tissue sample) is treated with a citrate buffer. Citrate buffer can be used for antigen retrieval to decrosslink antigens and fixation medium in the biological sample. Thus, any suitable decrosslinking agent can be used in addition to or alternatively to citrate buffer. In some embodiments, for example, the biological sample (e.g., a fixed frozen tissue sample) is decrosslinked with TE buffer.

In any of the foregoing, the biological sample can further be stained, imaged, and/or destained. For example, in some embodiments, a fresh frozen tissue sample or fixed frozen tissue sample is stained (e.g., via eosin and/or hematoxylin), imaged, destained (e.g., via HCl), or a combination thereof. In some embodiments, when a fresh frozen tissue sample is fixed in methanol, it is treated with isopropanol prior to being stained (e.g., via eosin and/or hematoxylin), imaged, destained (e.g., via HCl), or a combination thereof. In some embodiments when a fixed frozen tissue sample is treated with a sucrose gradient, it can be rehydrated with an ethanol gradient before being stained, (e.g., via eosin and/or hematoxylin), imaged, destained (e.g., via HCl), decrosslinked (e.g., via TE buffer or citrate buffer), or a combination thereof. In some embodiments, the biological sample can undergo further fixation (e.g., while mounted on a substrate), stained, imaged, and/or destained. For example, a fixed frozen biological sample may be subject to an additional fixing step (e.g., using PFA) before optional ethanol rehydration, staining, imaging, and/or destaining.

In any of the foregoing, the biological sample can be fixed using PAXgene. For example, the biological sample can be fixed using PAXgene in addition, or alternatively to, a fixative disclosed herein or known in the art (e.g., alcohol, acetone, acetone-alcohol, formalin, paraformaldehyde). PAXgene is a non-cross-linking mixture of different alcohols, acid and a soluble organic compound that preserves morphology and bio-molecules. It is a two-reagent fixative system in which tissue is firstly fixed in a solution containing methanol and acetic acid then stabilized in a solution containing ethanol. See, Ergin B. et al., J Proteome Res. 2010 Oct. 1; 9 (10): 5188-96; Kap M. et al., PLOS One.; 6 (11): e27704 (2011); and Mathieson W. et al., Am J Clin Pathol.; 146 (1): 25-40 (2016), each of which are hereby incorporated by reference in their entirety, for a description and evaluation of PAXgene for tissue fixation. Thus, in some embodiments, when the biological sample, e.g., the tissue sample, is fixed in a fixative including alcohol, the fixative is PAXgene. In some embodiments, a fresh frozen tissue sample is fixed with PAXgene. In some embodiments, a fixed frozen tissue sample is fixed with PAXgene.

In some embodiments, the biological sample, e.g., the tissue sample is fixed, for example in methanol, acetone, acetone-methanol, PFA, PAXgene or is formalin-fixed and paraffin-embedded (FFPE). In some embodiments, the biological sample comprises intact cells. In some embodiments, the biological sample is a cell pellet, e.g., a fixed cell pellet, e.g., an FFPE cell pellet. FFPE samples are used in some instances in the RTL methods disclosed herein. A limitation of direct RNA capture for fixed samples is that the RNA integrity of fixed (e.g., FFPE) samples can be lower than a fresh sample, thereby making it more difficult to capture RNA directly, e.g., by capture of a common sequence such as a poly(A) tail of an mRNA molecule. However, by utilizing RTL probes that hybridize to RNA target sequences in the transcriptome, one can avoid a requirement for RNA analytes to have both a poly(A) tail and target sequences intact. Accordingly, RTL probes can be utilized to beneficially improve capture and spatial analysis of fixed samples. The biological sample, e.g., tissue sample, can be stained, and imaged prior, during, and/or after each step of the methods described herein. Any of the methods described herein or known in the art can be used to stain and/or image the biological sample. In some embodiments, the imaging occurs prior to destaining the sample. In some embodiments, the biological sample is stained using an H & E staining method. In some embodiments, the tissue sample is stained and imaged for about 10 minutes to about 2 hours (or any of the subranges of this range described herein). Additional time may be needed for staining and imaging of different types of biological samples.

The tissue sample can be obtained from any suitable location in a tissue or organ of a subject, e.g., a human subject. In some instances, the sample is a mouse sample. In some instances, the sample is a human sample. In some embodiments, the sample can be derived from skin, brain, breast, lung, liver, kidney, prostate, tonsil, thymus, testes, bone, lymph node, ovary, eye, heart, or spleen. In some instances, the sample is a human or mouse breast tissue sample. In some instances, the sample is a human or mouse brain tissue sample. In some instances, the sample is a human or mouse lung tissue sample. In some instances, the sample is a human or mouse tonsil tissue sample. In some instances, the sample is a human or mouse liver tissue sample. In some instances, the sample is a human or mouse bone, skin, kidney, thymus, testes, or prostate tissue sample. In some embodiments, the tissue sample is derived from normal or diseased tissue. In some embodiments, the sample is an embryo sample. The embryo sample can be a non-human embryo sample. In some instances, the sample is a mouse embryo sample.

Biological samples are also described in Section (I)(d) of PCT Publication No. WO2020/176788 and/or U.S. Patent Application Publication No. 2020/0277663.

The following embodiments can be used with any of the methods described herein. In some embodiments, the biological sample (e.g., a fixed and/or stained biological sample) is imaged. In some embodiments, the biological sample is visualized or imaged using bright field microscopy. In some embodiments, the biological sample is visualized or imaged using fluorescence microscopy. Additional methods of visualization and imaging are known in the art. Non-limiting examples of visualization and imaging include expansion microscopy, bright field microscopy, dark field microscopy, phase contrast microscopy, electron microscopy, fluorescence microscopy, reflection microscopy, interference microscopy and confocal microscopy. In some embodiments, the sample is stained and imaged prior to adding reagents for analyzing captured analytes as disclosed herein to the biological sample.

In some embodiments, the methods include staining the biological sample. In some embodiments, the staining includes the use of hematoxylin and/or eosin. Non-limiting examples of stains include histological stains (e.g., hematoxylin and/or eosin) and immunological stains (e.g., fluorescent stains). In some embodiments, a biological sample can be stained using any number of biological stains, including but not limited to, acridine orange, Bismarck brown, carmine, coomassie blue, cresyl violet, DAPI, eosin, ethidium bromide, acid fuchsine, hematoxylin, Hoechst stains, iodine, methyl green, methylene blue, neutral red, Nile blue, Nile red, osmium tetroxide, propidium iodide, rhodamine, or safranin. In some instances, the biological sample can be stained using known staining techniques, including Can-Grunwald, Giemsa, hematoxylin and eosin (H & E), Jenner's, Leishman, Masson's trichrome, Papanicolaou, Romanowsky, silver, Sudan, Wright's, and/or Periodic Acid Schiff (PAS) staining techniques. PAS staining is typically performed after formalin or acetone fixation.

In some embodiments, the staining includes the use of a detectable label selected from the group consisting of a radioisotope, a fluorophore, a chemiluminescent compound, a bioluminescent compound, or a combination thereof.

In some embodiments, a biological sample is permeabilized with one or more permeabilization reagents. For example, permeabilization of a biological sample can facilitate analyte capture. Exemplary permeabilization agents and conditions are described in Section (I)(d)(ii)(13) or the Exemplary Embodiments Section of PCT Publication No. WO2020/176788 and/or U.S. Patent Application Publication No. 2020/0277663. Briefly, in any of the methods described herein, the method includes a step of permeabilizing the biological sample. For example, the biological sample can be permeabilized to facilitate transfer of the extension products to the capture probes on the array. In some embodiments, the permeabilizing includes the use of an organic solvent (e.g., acetone, ethanol, and methanol), a detergent (e.g., saponin, Triton X-100™, Tween-20™, or sodium dodecyl sulfate (SDS)), an enzyme (an endopeptidase, an exopeptidase, a protease), or combinations thereof. In some embodiments, the permeabilizing includes the use of an endopeptidase, a protease, SDS, polyethylene glycol tert-octylphenyl ether, polysorbate 80, polysorbate 20, N-lauroylsarcosine sodium salt solution, saponin, Triton X-100™, Tween-20™, or combinations thereof. In some embodiments, the endopeptidase is pepsin. In some embodiments, the endopeptidase is proteinase K. Additional methods for sample permeabilization are described, for example, in Jamur et al., Method Mol. Biol. 588:63-66, 2010, the entire contents of which are incorporated herein by reference.

Array-based spatial analysis methods can involve the transfer of one or more analytes or derivatives thereof from a biological sample to an array of features on a substrate, where each feature is associated with a unique spatial location on the array. Subsequent analysis of the transferred analytes includes determining the identity of the analytes and the spatial location of the analytes within the biological sample. The spatial location of an analyte within the biological sample is determined based on the feature to which the analyte is bound (e.g., directly or indirectly) on the array, and the feature's relative spatial location within the array.

A “capture probe” refers to any molecule capable of capturing (directly or indirectly) and/or labelling an analyte (e.g., an analyte of interest) in a biological sample. In some embodiments, the capture probe is a nucleic acid or a polypeptide. In some embodiments, the capture probe includes a barcode (e.g., a spatial barcode and/or a unique molecular identifier (UMI) and a capture domain). In some instances, the capture probe includes a homopolymer sequence, such as a poly(T) sequence. In some embodiments, a capture probe can include a cleavage domain and/or a functional domain (e.g., a primer-binding site, such as for next-generation sequencing (NGS)). See, e.g., Section (II)(b) (e.g., subsections (i)-(vi)) of PCT Publication No. WO2020/176788 and/or U.S. Patent Application Publication No. 2020/0277663. Generation of capture probes can be achieved by any appropriate method, including those described in Section (II)(d)(ii) of PCT Publication No. WO2020/176788 and/or U.S. Patent Application Publication No. 2020/0277663.

In some embodiments, more than one analyte type (e.g., nucleic acids and proteins) from a biological sample can be detected (e.g., simultaneously or sequentially) using any appropriate multiplexing technique, such as those described in Section (IV) of PCT Publication No. WO2020/176788 and/or U.S. Patent Application Publication No. 2020/0277663.

In some embodiments, detection of one or more analytes (e.g., protein analytes) can be performed using one or more analyte capture agents. As used herein, an “analyte capture agent” refers to an agent that interacts with an analyte (e.g., an analyte in a biological sample) and with a capture probe (e.g., a capture probe attached to a substrate or a feature) to identify the analyte.

In some embodiments, the analyte capture agent includes: (i) an analyte binding moiety (e.g., that binds to an analyte), for example, an antibody or antigen-binding fragment thereof; (ii) analyte binding moiety barcode; and (iii) an analyte capture sequence. As used herein, the term “analyte binding moiety barcode” refers to a barcode that is associated with or otherwise identifies the analyte binding moiety. As used herein, the term “analyte capture sequence” refers to a region or moiety configured to hybridize to, bind to, couple to, or otherwise interact with a capture domain of a capture probe. In some cases, an analyte binding moiety barcode (or portion thereof) may be able to be removed (e.g., cleaved) from the analyte capture agent. Additional description of analyte capture agents can be found in Section (II)(b)(ix) of PCT Publication No. WO2020/176788 and/or Section (II)(b)(viii) U.S. Patent Application Publication No. 2020/0277663.

In some instances, a capture probe and a nucleic acid analyte interaction (or any other nucleic acid to nucleic acid interaction) occurs because the sequences of the two nucleic acids are substantially complementary to one another. By “substantial,” “substantially” and the like, two nucleic acid sequences can be complementary when at least 60% of the nucleotide residues of one nucleic acid sequence are complementary to nucleotide residues in the other nucleic acid sequence. The complementary residues within a particular complementary nucleic acid sequence need not always be contiguous with each other, and can be interrupted by one or more non-complementary residues within the complementary nucleic acid sequence. In some embodiments, at least 60%, but less than 100%, of the residues of one of the two complementary nucleic acid sequences are complementary to residues in the other nucleic acid sequence. In some embodiments, at least 70%, 80%, 90%, 95% or 99% of the residues of one nucleic acid sequence are complementary to residues in the other nucleic acid sequence. Sequences are said to be “substantially complementary” when at least 60% (e.g., at least 70%, at least 80%, or at least 90%) of the residues of one nucleic acid sequence are complementary to residues in the other nucleic acid sequence. In some embodiments, the biological sample is mounted on a first substrate and the substrate comprising the array of capture probes is a second substrate. During this process, one or more analytes or analyte derivatives (e.g., intermediate agents; e.g., ligation products) are released from the biological sample and migrate to the second substrate comprising an array of capture probes. In some embodiments, the release and migration of the analytes or analyte derivatives to the second substrate comprising the array of capture probes occurs in a manner that preserves the original spatial context of the analytes in the biological sample. This method can be referred to as a sandwiching process, which is described e.g., in U.S. Patent Application Pub. No. 2021/0189475 and PCT Pub. Nos. WO 2021/252747 A1, WO 2022/061152 A2, and WO 2022/140028 A1.

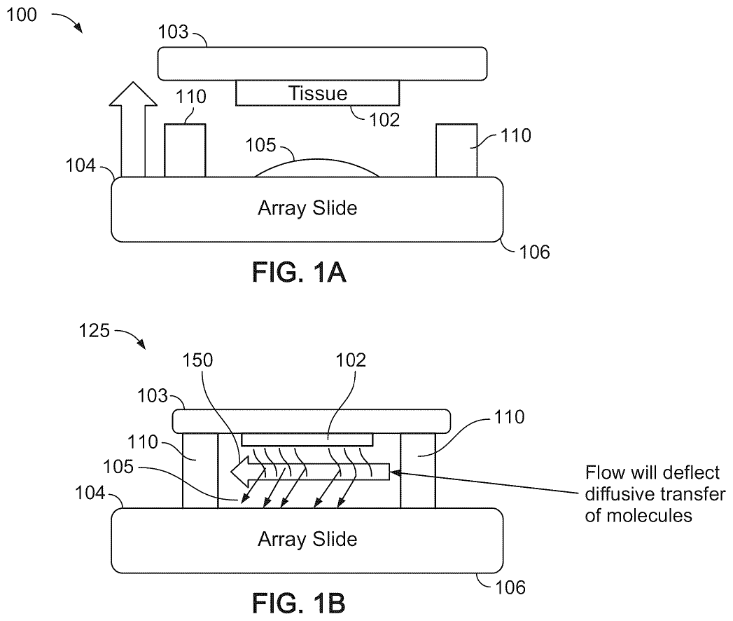

FIG. 1A shows an exemplary sandwiching process 100 where a first substrate (e.g., slide 103), including a biological sample 102, and a second substrate (e.g., array slide 104 including an array having spatially barcoded capture probes 106) are brought into proximity with one another. As shown in FIG. 1A a liquid reagent (e.g., permeabilization solution 105) is introduced on the second substrate in proximity to the capture probes 106 and in between the biological sample 102 and the second substrate (e.g., slide 104 including an array having spatially barcoded capture probes 106). The permeabilization solution 105 may release analytes or analyte derivatives (e.g., intermediate agents; e.g., ligation products) that can be captured by the capture probes of the array 106.

During the exemplary sandwiching process, the first substrate is aligned with the second substrate, such that at least a portion of the biological sample is aligned with at least a portion of the capture probes (e.g., aligned in a sandwich configuration). As shown, the second substrate (e.g., array slide 104) is in an inferior position to the first substrate (e.g., slide 103). In some embodiments, the first substrate (e.g., slide 103) may be positioned superior to the second substrate (e.g., slide 104). A reagent medium 105 within a gap between the first substrate (e.g., slide 103) and the second substrate (e.g., slide 104) creates a liquid interface between the two substrates. The reagent medium may be a permeabilization solution which permeabilizes and/or digests the biological sample 102. In some embodiments wherein the biological sample 102 has been pre-permeabilized, the reagent medium is not a permeabilization solution. Herein, the reagent medium may also comprise one or more of a monovalent salt, a divalent salt, ethylene carbonate, and/or glycerol. In some embodiments, analytes (e.g., mRNA transcripts) and/or analyte derivatives (e.g., intermediate agents; e.g., ligation products) of the biological sample 102 may release from the biological sample, and actively or passively migrate (e.g., diffuse) across the gap toward the capture probes on the array 106. Alternatively, in certain embodiments, migration of the analyte or analyte derivative (e.g., intermediate agent; e.g., ligation product) from the biological sample is performed actively (e.g., electrophoretic, by applying an electric field to promote migration). Exemplary methods of electrophoretic migration are described in WO 2020/176788, and US. Patent Application Pub. No. 2021/0189475, each of which is hereby incorporated by reference.

As further shown, one or more spacers 110 may be positioned between the first substrate (e.g., slide 103) and the second substrate (e.g., array slide 104 including spatially barcoded capture probes 106). The one or more spacers 110 may be configured to maintain a separation distance between the first substrate and the second substrate. While the one or more spacers 110 is shown as disposed on the second substrate, the spacer may additionally or alternatively be disposed on the first substrate.

In some embodiments, the one or more spacers 110 is configured to maintain a separation distance between first and second substrates that is between about 2 microns and 1 mm (e.g., between about 2 microns and 800 microns, between about 2 microns and 700 microns, between about 2 microns and 600 microns, between about 2 microns and 500 microns, between about 2 microns and 400 microns, between about 2 microns and 300 microns, between about 2 microns and 200 microns, between about 2 microns and 100 microns, between about 2 microns and 25 microns, or between about 2 microns and 10 microns), measured in a direction orthogonal to the surface of first substrate that supports the biological sample. In some instances, the separation distance is about 2, 3, 4, 5, 6, 7, 8, 9, 10, 11, 12, 13, 14, 15, 16, 17, 18, 19, 20, 21, 22, 23, 24, or 25 microns. In some embodiments, the separation distance is less than 50 microns. In some embodiments, the separation distance is less than 25 microns. In some embodiments, the separation distance is less than 20 microns. The separation distance may include a distance of at least 2 μm.

FIG. 1B shows a fully formed sandwich configuration 125 creating a chamber 150 formed from the one or more spacers 110, the first substrate (e.g., the slide 103), and the second substrate (e.g., the slide 104 including an array 106 having spatially barcoded capture probes) in accordance with some example implementations. In the example of FIG. 1B, the liquid reagent (e.g., the permeabilization solution 105) fills the volume of the chamber 150 and may create a permeabilization buffer that allows analytes (e.g., mRNA transcripts and/or other molecules) or analyte derivatives (e.g., intermediate agents; e.g., ligation products) to diffuse from the biological sample 102 toward the capture probes of the second substrate (e.g., slide 104). In some aspects, flow of the permeabilization buffer may deflect transcripts and/or molecules from the biological sample 102 and may affect diffusive transfer of analytes or analyte derivatives (e.g., intermediate agents; e.g., ligation products) for spatial analysis. A partially or fully sealed chamber 150 resulting from the one or more spacers 110, the first substrate, and the second substrate may reduce or prevent flow from undesirable convective movement of transcripts and/or molecules over the diffusive transfer from the biological sample 102 to the capture probes.

The sandwiching process methods described above can be implemented using a variety of hardware components. For example, the sandwiching process methods can be implemented using a sample holder (also referred to herein as a support device, a sample handling apparatus, and an array alignment device). Further details on support devices, sample holders, sample handling apparatuses, or systems for implementing a sandwiching process are described in, e.g., US. Patent Application Pub. No. 2021/0189475, and PCT Publ. No. WO 2022/061152 A2, each of which are incorporated by reference in their entirety.

In some embodiments of a sample holder, the sample holder can include a first member including a first retaining mechanism configured to retain a first substrate comprising a biological sample. The first retaining mechanism can be configured to retain the first substrate disposed in a first plane. The sample holder can further include a second member including a second retaining mechanism configured to retain a second substrate disposed in a second plane. The sample holder can further include an alignment mechanism connected to one or both of the first member and the second member. The alignment mechanism can be configured to align the first and second members along the first plane and/or the second plane such that the sample contacts at least a portion of the reagent medium when the first and second members are aligned and within a threshold distance along an axis orthogonal to the second plane. The adjustment mechanism may be configured to move the second member along the axis orthogonal to the second plane and/or move the first member along an axis orthogonal to the first plane.

In some embodiments, the adjustment mechanism includes a linear actuator. In some embodiments, the linear actuator is configured to move the second member along an axis orthogonal to the plane of the first member and/or the second member. In some embodiments, the linear actuator is configured to move the first member along an axis orthogonal to the plane of the first member and/or the second member. In some embodiments, the linear actuator is configured to move the first member, the second member, or both the first member and the second member at a velocity of at least 0.1 mm/sec. In some embodiments, the linear actuator is configured to move the first member, the second member, or both the first member and the second member with an amount of force of at least 0.1 lbs.

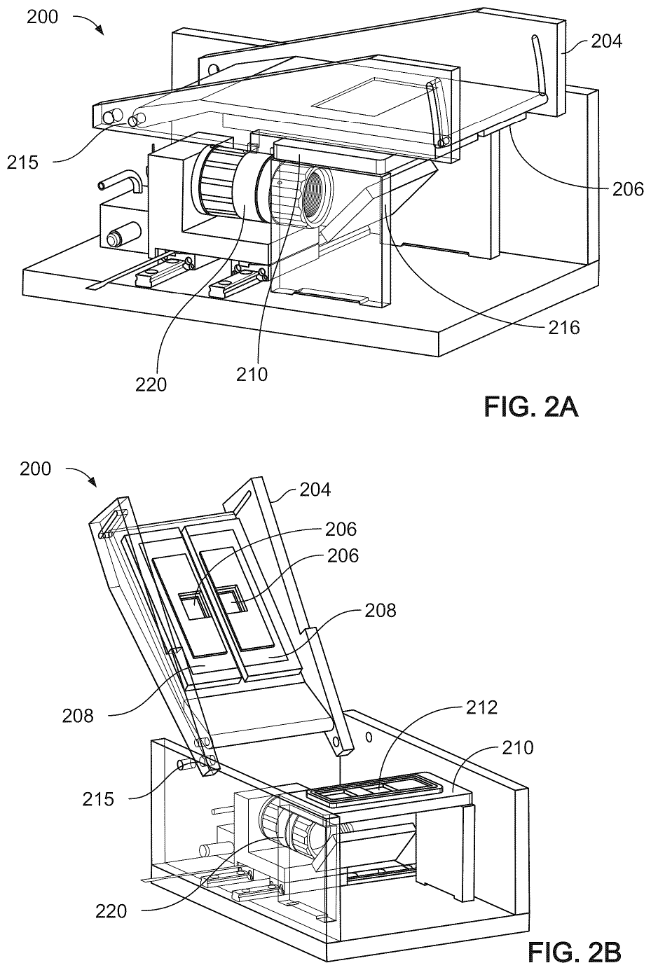

FIG. 2A is a perspective view of an example sample handling apparatus 200 in a closed position in accordance with some example implementations. As shown, the sample handling apparatus 200 includes a first member 204, a second member 210, optionally an image capture device 220, a first substrate 206, optionally a hinge 215, and optionally a mirror 216. The hinge 215 may be configured to allow the first member 204 to be positioned in an open or closed configuration by opening and/or closing the first member 204 in a clamshell manner along the hinge 215.

FIG. 2B is a perspective view of the example sample handling apparatus 200 in an open position in accordance with some example implementations. As shown, the sample handling apparatus 200 includes one or more first retaining mechanisms 208 configured to retain one or more first substrates 206. In the example of FIG. 2B, the first member 204 is configured to retain two first substrates 206, however the first member 204 may be configured to retain more or fewer first substrates 206.

In some aspects, when the sample handling apparatus 200 is in an open position (e.g., in FIG. 2B), the first substrate 206 and/or the second substrate 212 may be loaded and positioned within the sample handling apparatus 200 such as within the first member 204 and the second member 210, respectively. As noted, the hinge 215 may allow the first member 204 to close over the second member 210 and form a sandwich configuration.

In some aspects, after the first member 204 closes over the second member 210, an adjustment mechanism of the sample handling apparatus 200 may actuate the first member 204 and/or the second member 210 to form the sandwich configuration for the permeabilization step (e.g., bringing the first substrate 206 and the second substrate 212 closer to each other and within a threshold distance for the sandwich configuration). The adjustment mechanism may be configured to control a speed, an angle, a force, or the like of the sandwich configuration.

In some embodiments, the biological sample (e.g., sample 102 from FIG. 1A) may be aligned within the first member 204 (e.g., via the first retaining mechanism 208) prior to closing the first member 204 such that a desired region of interest of the sample is aligned with the barcoded array of the second substrate (e.g., the slide 104 from FIG. 1A), e.g., when the first and second substrates are aligned in the sandwich configuration. Such alignment may be accomplished manually (e.g., by a user) or automatically (e.g., via an automated alignment mechanism). After or before alignment, spacers may be applied to the first substrate 206 and/or the second substrate 212 to maintain a minimum spacing between the first substrate 206 and the second substrate 212 during sandwiching. In some aspects, the permeabilization solution (e.g., permeabilization solution 305) may be applied to the first substrate 206 and/or the second substrate 212. The first member 204 may then close over the second member 210 and form the sandwich configuration. Analytes or analyte derivatives (e.g., intermediate agents; e.g., ligation products) may be captured by the capture probes of the array and may be processed for spatial analysis.

In some embodiments, during the permeabilization step, the image capture device 220 may capture images of the overlap area between the biological sample and the capture probes on the array 106. If more than one first substrates 206 and/or second substrates 212 are present within the sample handling apparatus 200, the image capture device 220 may be configured to capture one or more images of one or more overlap areas.

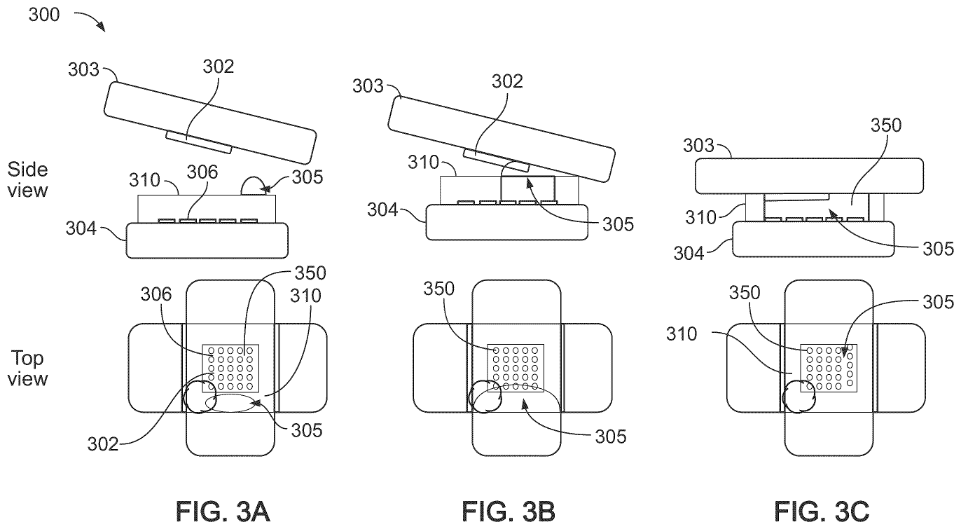

Provided herein are methods for delivering a fluid to a biological sample disposed on an area of a first substrate and an array disposed on a second substrate. FIGS. 3A-3C depict a side view and a top view of an exemplary angled closure workflow 300 for sandwiching a first substrate (e.g., slide 303) having a biological sample 302 and a second substrate (e.g., slide 304 having capture probes 306) in accordance with some exemplary implementations.

FIG. 3A depicts the first substrate (e.g., the slide 303 including a biological sample 302) angled over (superior to) the second substrate (e.g., slide 304). As shown, reagent medium (e.g., permeabilization solution) 305 is located on the spacer 310 toward the right-hand side of the side view in FIG. 3A. While FIG. 3A depicts the reagent medium on the right hand side of side view, it should be understood that such depiction is not meant to be limiting as to the location of the reagent medium on the spacer.

FIG. 3B shows that as the first substrate lowers, and/or as the second substrate rises, the dropped side of the first substrate (e.g., a side of the slide 303 angled toward the second substrate) may contact the reagent medium 305. The dropped side of the first substrate may urge the reagent medium 305 toward the opposite direction (e.g., towards an opposite side of the spacer 310, towards an opposite side of the first substrate relative to the dropped side). For example, in the side view of FIG. 3B the reagent medium 305 may be urged from right to left as the sandwich is formed.

In some embodiments, the first substrate and/or the second substrate are further moved to achieve an approximately parallel arrangement of the first substrate and the second substrate.

FIG. 3C depicts a full closure of the sandwich between the first substrate and the second substrate with the spacer 310 contacting both the first substrate and the second substrate and maintaining a separation distance and optionally the approximately parallel arrangement between the two substrates. As shown in the top view of FIG. 3C, the spacer 310 fully encloses and surrounds the biological sample 302 and the capture probes 306, and the spacer 310 form the sides of chamber 350 which holds a volume of the reagent medium 305.

While FIG. 3C depicts the first substrate (e.g., the slide 303 including biological sample 302) angled over (superior to) the second substrate (e.g., slide 304) and the second substrate comprising the spacer 310, it should be understood that an exemplary angled closure workflow can include the second substrate angled over (superior to) the first substrate and the first substrate comprising the spacer 310.

It may be desirable that the reagent medium be free from air bubbles between the substrates to facilitate transfer of target analytes with spatial information. Additionally, air bubbles present between the substrates may obscure at least a portion of an image capture of a desired region of interest. Accordingly, it may be desirable to ensure or encourage suppression and/or elimination of air bubbles between the two substrates (e.g., slide 303 and slide 304) during a permeabilization step (e.g., step 104). In some aspects, it may be possible to reduce or eliminate bubble formation between the substrates using a variety of filling methods and/or closing methods. In some instances, the first substrate and the second substrate are arranged in an angled sandwich assembly as described herein. For example, during the sandwiching of the two substrates (e.g., the slide 303 and the slide 304), an angled closure workflow may be used to suppress or eliminate bubble formation.

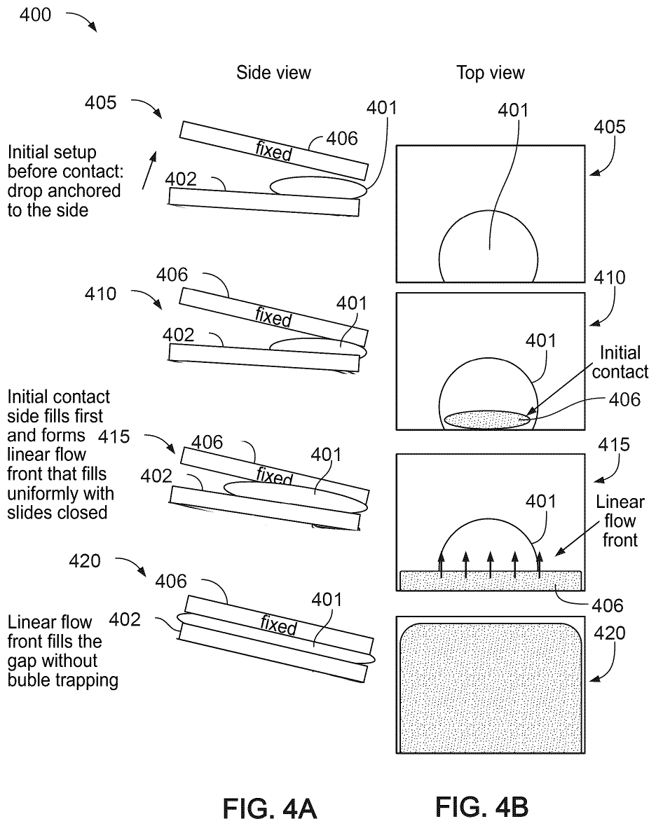

FIG. 4A is a side view of the angled closure workflow 400 in accordance with some exemplary implementations. FIG. 4B is a top view of the angled closure workflow 400 in accordance with some exemplary implementations. As shown at 405, reagent medium 401 is positioned to the side of the substrate 402 contacting the spring.

At step 410, the dropped side of the angled substrate 406 contacts the reagent medium 401 first. The contact of the substrate 406 with the reagent medium 401 may form a linear or low curvature flow front that fills uniformly with the slides closed.

At step 415, the substrate 406 is further lowered toward the substrate 402 (or the substrate 402 is raised up toward the substrate 406) and the dropped side of the substrate 406 may contact and may urge the reagent medium toward the side opposite the dropped side and creating a linear or low curvature flow front that may prevent or reduce bubble trapping between the substrates.

At step 420, the reagent medium 401 fills the gap between the substrate 406 and the substrate 402. The linear flow front of the liquid reagent may form by squeezing the 401 volume along the contact side of the substrate 402 and/or the substrate 406. Additionally, capillary flow may also contribute to filling the gap area.

In some embodiments, the reagent medium (e.g., 105 in FIG. 1A) comprises a permeabilization agent. In some embodiments, following initial contact between the biological sample and a permeabilization agent, the permeabilization agent can be removed from contact with the biological sample (e.g., by opening the sample holder). Suitable agents for this purpose include, but are not limited to, organic solvents (e.g., acetone, ethanol, and methanol), cross-linking agents (e.g., paraformaldehyde), detergents (e.g., saponin, Triton X-100™, Tween-20™, or sodium dodecyl sulfate (SDS)), and enzymes (e.g., trypsin, proteases (e.g., proteinase K). In some embodiments, the detergent is an anionic detergent (e.g., SDS or N-lauroylsarcosine sodium salt solution).

In some embodiments, the reagent medium comprises a lysis reagent. Lysis solutions can include ionic surfactants such as, for example, sarkosyl and sodium dodecyl sulfate (SDS). More generally, chemical lysis agents can include, without limitation, organic solvents, chelating agents, detergents, surfactants, and chaotropic agents. In some embodiments, the reagent medium comprises a protease. Exemplary proteases include, e.g., pepsin, trypsin, elastase, and proteinase K. In some embodiments, the reagent medium comprises a nuclease. In some embodiments, the nuclease comprises an RNase. In some embodiments, the RNase is selected from RNase A, RNase C, RNase H, and RNase I. In some embodiments, the reagent medium comprises one or more of sodium dodecyl sulfate (SDS) or a sodium salt thereof, proteinase K, pepsin, N-lauroylsarcosine, and RNase.

In some embodiments, the reagent medium comprises polyethylene glycol (PEG). In some embodiments, the PEG is from about 2 K to about 16 K. In some embodiments, the PEG is PEG 2 K, 3 K, 4 K, 5 K, 6 K, 7 K, 8 K, 9 K, 10 K, 11 K, 12 K, 13 K, 14 K, 15 K, or 16 K. In some embodiments, the PEG is present at a concentration from about 2% to 25%, from about 4% to about 23%, from about 6% to about 21%, or from about 8% to about 20% (v/v).

In certain embodiments, a dried permeabilization reagent is applied or formed as a layer on the first substrate or the second substrate or both prior to contacting the biological sample with the array. For example, a permeabilization reagent can be deposited in solution on the first substrate or the second substrate or both and then dried.

In some instances, the aligned portions of the biological sample and the array are in contact with the reagent medium for about 1 minute, about 5 minutes, about 10 minutes, about 12 minutes, about 15 minutes, about 18 minutes, about 20 minutes, about 25 minutes, about 30 minutes, about 36 minutes, about 45 minutes, or about an hour. In some instances, the aligned portions of the biological sample and the array are in contact with the reagent medium for about 1-60 minutes.

In some instances, the device is configured to control a temperature of the first and second substrates. In some embodiments, the temperature of the first and second members is lowered to a first temperature that is below room temperature.

There are at least two methods to associate a spatial barcode with one or more neighboring cells, such that the spatial barcode identifies the one or more cells, and/or contents of the one or more cells, as associated with a particular spatial location. One method is to promote analytes or analyte proxies (e.g., intermediate agents) out of a cell and towards a spatially-barcoded array (e.g., including spatially-barcoded capture probes). Another method is to cleave spatially-barcoded capture probes from an array and promote the spatially-barcoded capture probes towards and/or into or onto the biological sample.

In some cases, capture probes may be configured to prime, replicate, and consequently yield optionally barcoded extension products from a template (e.g., a DNA or RNA template, such as an analyte or an intermediate agent (e.g., a ligation product or an analyte capture agent), or a portion thereof), or derivatives thereof (see, e.g., Section (II)(b)(vii) of PCT Publication No. WO2020/176788 and/or U.S. Patent Application Publication No. 2020/0277663 regarding extended capture probes). In some cases, capture probes may be configured to form ligation products with a template (e.g., a DNA or RNA template, such as an analyte or an intermediate agent, or portion thereof), thereby creating ligation products that serve as proxies for the template.

As used herein, an “extended capture probe” refers to a capture probe having additional nucleotides added to the terminus (e.g., 3′ or 5′ end) of the capture probe thereby extending the overall length of the capture probe. For example, an “extended 3′ end” indicates additional nucleotides were added to the most 3′ nucleotide of the capture probe to extend the length of the capture probe, for example, by polymerization reactions used to extend nucleic acid molecules including templated polymerization catalyzed by a polymerase (e.g., a DNA polymerase or a reverse transcriptase). In some embodiments, extending the capture probe includes adding to a 3′ end of a capture probe a nucleic acid sequence that is complementary to a nucleic acid sequence of an analyte or intermediate agent specifically bound to the capture domain of the capture probe. In some embodiments, the capture probe is extended by a reverse transcriptase. In some embodiments, the capture probe is extended using one or more DNA polymerases. In some embodiments, the extended capture probes include the sequence of the capture domain, the sequence of the spatial barcode of the capture probe, and the complementary sequence of the template used for extension of the capture probe.

In some embodiments, extended capture probes are amplified (e.g., in bulk solution or on the array) to yield quantities that are sufficient for downstream analysis, e.g., sequencing. In some embodiments, extended capture probes (e.g., DNA molecules) can act as templates for an amplification reaction (e.g., a polymerase chain reaction).

Additional variants of spatial analysis methods, including in some embodiments, an imaging step, are described in Section (II)(a) of PCT Publication No. WO2020/176788 and/or U.S. Patent Application Publication No. 2020/0277663. Analysis of captured analytes (and/or intermediate agents or portions thereof), for example, including sample removal, extension of capture probes using the capture analyte as a template, sequencing (e.g., of a cleaved extended capture probe and/or a cDNA molecule complementary to an extended capture probe), sequencing on the array (e.g., using, for example, in situ hybridization or in situ ligation approaches), temporal analysis, and/or proximity capture, is described in Section (II)(g) of PCT Publication No. WO2020/176788 and/or U.S. Patent Application Publication No. 2020/0277663. Some quality control measures are described in Section (II)(h) of PCT Publication No. WO2020/176788 and/or U.S. Patent Application Publication No. 2020/0277663.

Spatial information can provide information of biological and/or medical importance. For example, the methods and compositions described herein can allow for: identification of one or more biomarkers (e.g., diagnostic, prognostic, and/or for determination of efficacy of a treatment) of a disease or disorder; identification of a candidate drug target for treatment of a disease or disorder; identification (e.g., diagnosis) of a subject as having a disease or disorder; identification of stage and/or prognosis of a disease or disorder in a subject; identification of a subject as having an increased likelihood of developing a disease or disorder; monitoring of progression of a disease or disorder in a subject; determination of efficacy of a treatment of a disease or disorder in a subject; identification of a patient subpopulation for which a treatment is effective for a disease or disorder; modification of a treatment of a subject with a disease or disorder; selection of a subject for participation in a clinical trial; and/or selection of a treatment for a subject with a disease or disorder. Exemplary methods for identifying spatial information of biological and/or medical importance can be found in U.S. Patent Application Publication Nos. 2021/0140982, 2021/0198741, and 2021/0199660.

Spatial information can provide information of biological importance. For example, the methods and compositions described herein can allow for: identification of transcriptome and/or proteome expression profiles (e.g., in healthy and/or diseased tissue); identification of multiple analyte types in close proximity (e.g., nearest neighbor or proximity based analysis); determination of up- and/or down-regulated genes and/or proteins in diseased tissue; characterization of tumor microenvironments; characterization of tumor immune responses; characterization of cells types and their co-localization in healthy and diseased tissue; and identification of genetic variants within tissues (e.g., based on gene and/or protein expression profiles associated with specific disease or disorder biomarkers).

Typically, for spatial array-based methods, a substrate functions as a support for direct or indirect attachment of capture probes to features of the array. A “feature” is an entity that acts as a support or repository for various molecular entities used in spatial analysis. In some embodiments, some or all of the features in an array are functionalized for analyte capture. Exemplary substrates are described in Section (II)(c) of PCT Publication No. WO2020/176788 and/or U.S. Patent Application Publication No. 2020/0277663. Exemplary features and geometric attributes of an array can be found in Sections (II)(d)(i), (II)(d)(iii), and (II)(d)(iv) of PCT Publication No. WO2020/176788 and/or U.S. Patent Application Publication No. 2020/0277663.

Generally, analytes and/or intermediate agents (or portions thereof) can be captured when contacting a biological sample with a substrate including capture probes (e.g., a substrate with capture probes embedded, spotted, printed, fabricated on the substrate, or a substrate with features (e.g., beads, wells) comprising capture probes). As used herein, “contact,” “contacted,” and/or “contacting,” a biological sample with a substrate refers to any contact (e.g., direct or indirect) such that capture probes can interact (e.g., bind covalently or non-covalently (e.g., hybridize)) with analytes from the biological sample. Capture can be achieved actively (e.g., using electrophoresis) or passively (e.g., using diffusion). Analyte capture is further described in Section (II)(e) of PCT Publication No. WO2020/176788 and/or U.S. Patent Application Publication No. 2020/0277663.

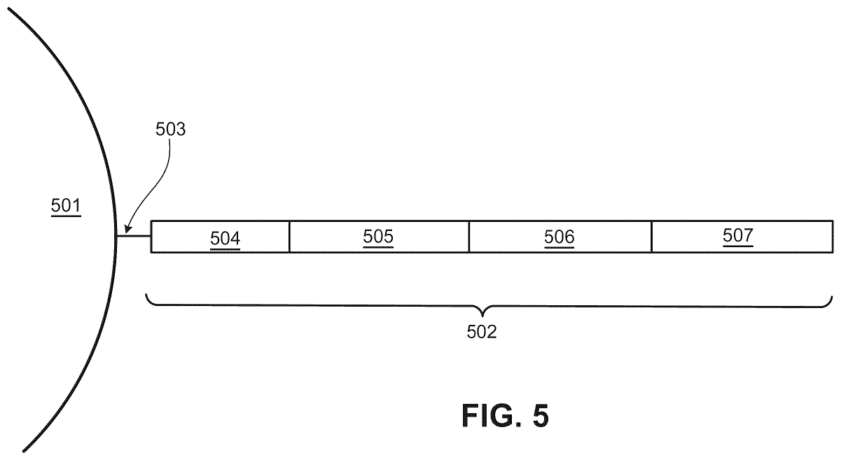

FIG. 5 is a schematic diagram showing an exemplary capture probe, as described herein. As shown, the capture probe 502 is optionally coupled to a feature 501 by a cleavage domain 503, such as a disulfide linker. The capture probe can include a functional sequence 504 that is useful for subsequent processing. The functional sequence 504 can include all or a part of sequencer specific flow cell attachment sequence (e.g., a P5 or P7 sequence), all or a part of a sequencing primer sequence, (e.g., a R1 primer binding site, a R2 primer binding site), or combinations thereof. The capture probe can also include a spatial barcode 505. The capture probe can also include a unique molecular identifier (UMI) sequence 506. While FIG. 5 shows the spatial barcode 505 as being located upstream (5′) of UMI sequence 506, it is to be understood that capture probes wherein UMI sequence 506 is located upstream (5′) of the spatial barcode 505 is also suitable for use in any of the methods described herein. The capture probe can also include a capture domain 507 to facilitate capture of a target analyte. The capture domain can have a sequence complementary to a sequence of a nucleic acid analyte. The capture domain can have a sequence complementary to a connected probe described herein. The capture domain can have a sequence complementary to an analyte capture sequence present in an analyte capture agent. The capture domain can have a sequence complementary to a splint oligonucleotide. A splint oligonucleotide, in addition to having a sequence complementary to a capture domain of a capture probe, can have a sequence complementary to a sequence of a nucleic acid analyte, a portion of a connected probe described herein, a capture handle sequence described herein, and/or a methylated adaptor described herein.

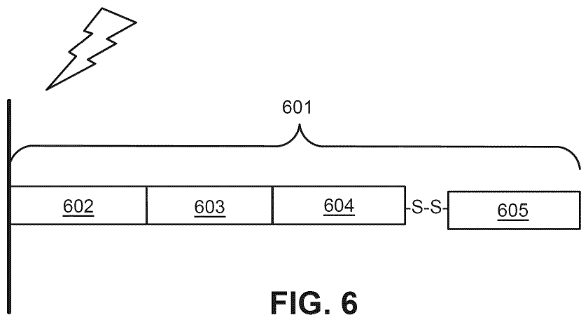

FIG. 6 is a schematic illustrating a cleavable capture probe, wherein the cleaved capture probe can enter into a non-permeabilized cell and bind to analytes within the sample. The capture probe 601 contains a cleavage domain 602, a cell penetrating peptide 603, a reporter molecule 604, and a disulfide bond (—S—S—). 605 represents all other parts of a capture probe, for example a spatial barcode and a capture domain.

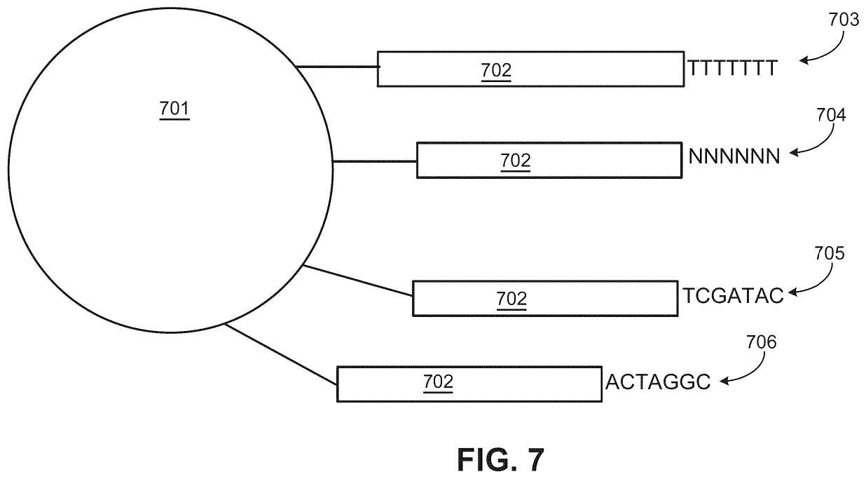

FIG. 7 is a schematic diagram of an exemplary multiplexed spatially-barcoded feature. In FIG. 7, the feature 701 can be coupled to spatially-barcoded capture probes, wherein the spatially-barcoded probes of a particular feature can possess the same spatial barcode, but have different capture domains designed to associate the spatial barcode of the feature with more than one target analyte. For example, a feature may include four different types of spatially-barcoded capture probes, each type of spatially-barcoded capture probe possessing the spatial barcode 702. One type of capture probe associated with the feature includes the spatial barcode 702 in combination with a poly(T) capture domain 703, designed to capture mRNA target analytes. A second type of capture probe associated with the feature includes the spatial barcode 702 in combination with a random N-mer capture domain 704 for gDNA analysis. A third type of capture probe associated with the feature includes the spatial barcode 702 in combination with a capture domain complementary to the analyte capture agent of interest 705. A fourth type of capture probe associated with the feature includes the spatial barcode 702 in combination with a capture probe that can specifically bind a nucleic acid molecule 706 that can function in a CRISPR assay (e.g., CRISPR/Cas9). While only four different capture probe-barcoded constructs are shown in FIG. 7, capture-probe barcoded constructs can be tailored for analyses of any given analyte associated with a nucleic acid and capable of binding with such a construct. For example, the schemes shown in FIG. 7 can also be used for concurrent analysis of other analytes disclosed herein, including, but not limited to: (a) mRNA, a lineage tracing construct, cell surface or intracellular proteins and metabolites, and gDNA; (b) mRNA, accessible chromatin (e.g., ATAC-seq, DNase-seq, and/or MNase-seq) cell surface or intracellular proteins and metabolites, and a perturbation agent (e.g., a CRISPR crRNA/sgRNA, TALEN, zinc finger nuclease, and/or antisense oligonucleotide as described herein); (c) mRNA, cell surface or intracellular proteins and/or metabolites, a barcoded labelling agent (e.g., the MHC multimers described herein), and a V (D) J sequence of an immune cell receptor (e.g., T-cell receptor). In some embodiments, a perturbation agent can be a small molecule, an antibody, a drug, an aptamer, a miRNA, a physical environmental (e.g., temperature change), or any other known perturbation agents. See, e.g., Section (II)(b) (e.g., subsections (i)-(vi)) of WO 2020/176788 and/or U.S. Patent Application Publication No. 2020/0277663. Generation of capture probes can be achieved by any appropriate method, including those described in Section (II)(d)(ii) of WO 2020/176788 and/or U.S. Patent Application Publication No. 2020/0277663.

The functional sequences can generally be selected for compatibility with any of a variety of different sequencing systems, e.g., Ion Torrent Proton or PGM, Illumina sequencing instruments, PacBio, Oxford Nanopore, etc., and the requirements thereof. In some embodiments, functional sequences can be selected for compatibility with non-commercialized sequencing systems. Examples of such sequencing systems and techniques, for which suitable functional sequences can be used, include (but are not limited to) Ion Torrent Proton or PGM sequencing, Illumina sequencing, PacBio SMRT sequencing, and Oxford Nanopore sequencing. Further, in some embodiments, functional sequences can be selected for compatibility with other sequencing systems, including non-commercialized sequencing systems.

In some embodiments, the spatial barcode 505 and functional sequences 504 are common to all of the probes attached to a given feature. In some embodiments, the UMI sequence 506 of a capture probe attached to a given feature is different from the UMI sequence of a different capture probe attached to the given feature.

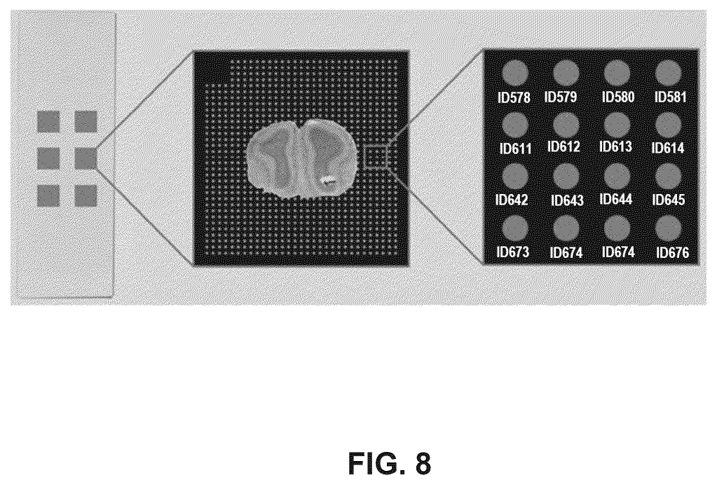

FIG. 8 depicts an exemplary arrangement of barcoded features within an array. From left to right, FIG. 8 shows (L) a slide including six spatially-barcoded arrays, (C) an enlarged schematic of one of the six spatially-barcoded arrays, showing a grid of barcoded features in relation to a biological sample, and (R) an enlarged schematic of one section of an array, showing the specific identification of multiple features within the array (labelled as ID578, ID579, ID560, etc.).

In some embodiments, more than one analyte type (e.g., nucleic acids and proteins) from a biological sample can be detected (e.g., simultaneously or sequentially) using any appropriate multiplexing technique, such as those described in Section (IV) of PCT Publication No. WO2020/176788 and/or U.S. Patent Application Publication No. 2020/0277663.

In some cases, spatial analysis can be performed by attaching and/or introducing a molecule (e.g., a peptide, a lipid, or a nucleic acid molecule) having a barcode (e.g., a spatial barcode) to a biological sample (e.g., to a cell in a biological sample). In some embodiments, a plurality of molecules (e.g., a plurality of nucleic acid molecules) having a plurality of barcodes (e.g., a plurality of spatial barcodes) are introduced to a biological sample (e.g., to a plurality of cells in a biological sample) for use in spatial analysis. In some embodiments, after attaching and/or introducing a molecule having a barcode to a biological sample, the biological sample can be physically separated (e.g., dissociated) into single cells or cell groups for analysis. Some such methods of spatial analysis are described in Section (III) of PCT Publication No. WO2020/176788 and/or U.S. Patent Application Publication No. 2020/0277663.

In some cases, spatial analysis can be performed by detecting multiple oligonucleotides that hybridize to an analyte. In some instances, for example, spatial analysis can be performed using RNA-templated ligation (RTL). Methods of RTL have been described previously. See, e.g., Credle et al., Nucleic Acids Res. 2017 Aug. 21; 45 (14): e128. Typically, RTL includes hybridization of two oligonucleotides to adjacent sequences on an analyte (e.g., an RNA molecule, such as an mRNA molecule). In some instances, the oligonucleotides are DNA molecules. In some instances, one of the oligonucleotides includes at least two ribonucleic acid bases at the 3′ end and/or the other oligonucleotide includes a phosphorylated nucleotide at the 5′ end. In some instances, one of the two oligonucleotides includes a capture domain (e.g., a poly(A) sequence, a non-homopolymeric sequence). After hybridization to the analyte, a ligase (e.g., a T4 RNA ligase (Rnl2), a PBCV-1 DNA ligase, a Chlorella virus DNA ligase, a single-stranded DNA ligase, or a T4 DNA ligase) ligates the two oligonucleotides together, creating a ligation product. In some instances, the two oligonucleotides hybridize to sequences that are not adjacent to one another. For example, hybridization of the two oligonucleotides creates a gap between the hybridized oligonucleotides. In some instances, a polymerase (e.g., a DNA polymerase) can extend one of the oligonucleotides prior to ligation. After ligation, the ligation product is released from the analyte. In some instances, the ligation product is released using an endonuclease (e.g., RNase H). In some instances, the ligation product is removed using heat. In some instances, the ligation product is removed using KOH. The released ligation product can then be captured by capture probes (e.g., instead of direct capture of an analyte) on an array, optionally amplified, and sequenced, thus determining the location and optionally the abundance of the analyte in the biological sample.

In some instances, one or both of the oligonucleotides may hybridize to genomic DNA (gDNA) which can lead to false positive sequencing data from ligation events on gDNA (off target) in addition to the desired (on target) ligation events on target nucleic acids, (e.g., mRNA). Thus, in some embodiments, the disclosed methods can include contacting the biological sample with a deoxyribonuclease (DNase). The DNase can be an endonuclease or exonuclease. In some embodiments, the DNase digests single- and/or double-stranded DNA. Suitable DNases include, without limitation, a DNase I and a DNase II. Use of a DNase as described can mitigate false positive sequencing data from off target gDNA ligation events.

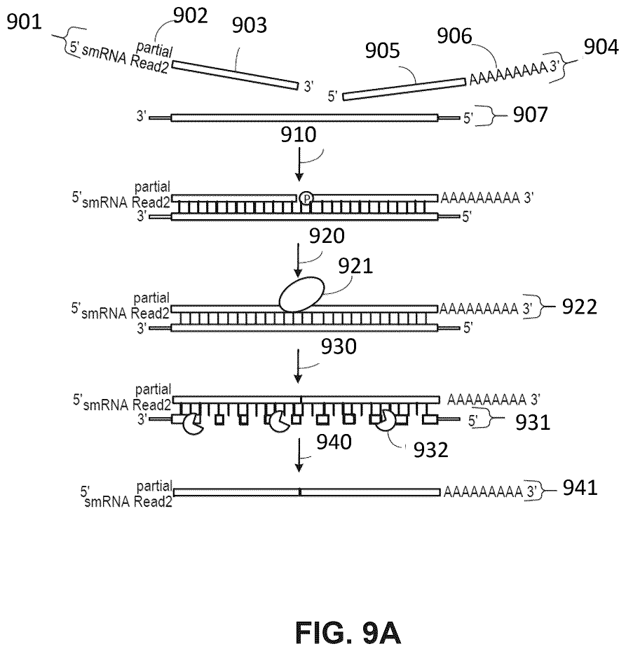

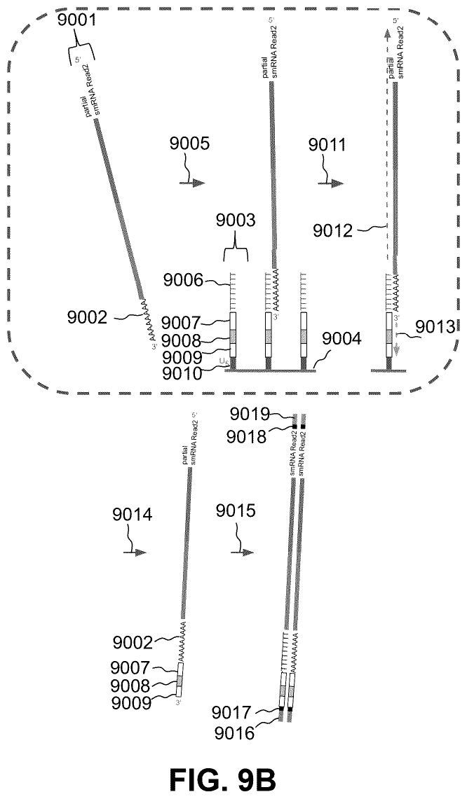

A non-limiting example of templated ligation methods disclosed herein is depicted in FIG. 9A. After a biological sample is contacted with a substrate including a plurality of capture probes and contacted with (a) a first probe 901 having a target-hybridization sequence 903 and a primer sequence 902 and (b) a second probe 904 having a target-hybridization sequence 905 and a capture domain (e.g., a poly-A sequence) 906, the first probe 901 and a second probe 904 hybridize 910 to an analyte 907. A ligase 921 ligates 920 the first probe to the second probe thereby generating a ligation product 922. The ligation product is released 930 from the analyte 931 by digesting the analyte using an endoribonuclease 932. The sample is permeabilized 940 and the ligation product 941 is able to hybridize to a capture probe on the substrate. Methods and composition for spatial detection using templated ligation have been described in PCT Publ. No. WO 2021/133849 A1, U.S. Pat. Nos. 11,332,790 and 11,505,828, each of which is incorporated by reference in its entirety.

In some embodiments, as shown in FIG. 9B, the ligation product 9001 includes a capture probe capture domain 9002, which can bind to a capture probe 9003 (e.g., a capture probe immobilized, directly or indirectly, on a substrate 9004). In some embodiments, methods provided herein include contacting 9005 a biological sample with a substrate 9004, wherein the capture probe 9003 is affixed to the substrate (e.g., immobilized to the substrate, directly or indirectly). In some embodiments, the capture probe capture domain 9002 of the ligation product specifically binds to the capture domain 9006. The capture probe can also include a unique molecular identifier (UMI) 9007, a spatial barcode 9008, a functional sequence 9009, and a cleavage domain 9010.

In some embodiments, methods provided herein include permeabilization of the biological sample such that the capture probe can more easily capture the ligation products (i.e., compared to no permeabilization). In some embodiments, polymerization reagents can be added to permeabilized biological samples. Incubation with the polymerization reagents can extend the capture probes 9011 to produce spatially-barcoded full-length cDNA 9012 and 9013 from the captured ligation products.

In some embodiments, the extended ligation products can be denatured 9014 from the capture probe and transferred (e.g., to a clean tube) for amplification, and/or library construction. The spatially-barcoded ligation products can be amplified 9015 via PCR prior to library construction. P5 9016, i5 9017, i7 9018, and P7 9019, and can be used as sample indexes. The amplicons can then be sequenced using paired-end sequencing using TruSeq Read 1 and TruSeq Read 2 as sequencing primer sites.

During analysis of spatial information, sequence information for a spatial barcode associated with an analyte is obtained, and the sequence information can be used to provide information about the spatial distribution of the analyte in the biological sample. Various methods can be used to obtain the spatial information. In some embodiments, specific capture probes and the analytes they capture are associated with specific locations in an array of features on a substrate. For example, specific spatial barcodes can be associated with specific array locations prior to array fabrication, and the sequences of the spatial barcodes can be stored (e.g., in a database) along with specific array location information, so that each spatial barcode uniquely maps to a particular array location.

Alternatively, specific spatial barcodes can be deposited at predetermined locations in an array of features during fabrication such that at each location, only one type of spatial barcode is present so that spatial barcodes are uniquely associated with a single feature of the array. Where necessary, the arrays can be decoded using any of the methods described herein so that spatial barcodes are uniquely associated with array feature locations, and this mapping can be stored as described above.

When sequence information is obtained for capture probes and/or analytes during analysis of spatial information, the locations of the capture probes and/or analytes can be determined by referring to the stored information that uniquely associates each spatial barcode with an array feature location. In this manner, specific capture probes and captured analytes are associated with specific locations in the array of features. Each array feature location represents a position relative to a coordinate reference point (e.g., an array location, a fiducial marker) for the array. Accordingly, each feature location has an “address” or location in the coordinate space of the array.

Some exemplary spatial analysis workflows are described in the Exemplary Embodiments section of PCT Publication No. WO2020/176788 and/or U.S. Patent Application Publication No. 2020/0277663. See, for example, the Exemplary embodiment starting with “In some non-limiting examples of the workflows described herein, the sample can be immersed . . . ” of PCT Publication No. WO2020/176788 and/or U.S. Patent Application Publication No. 2020/0277663. See also, e.g., the Visium Spatial Gene Expression Reagent Kits User Guide (e.g., Rev F, dated January 2022); and/or the Visium Spatial Gene Expression Reagent Kits-Tissue Optimization User Guide (e.g., Rev E, dated February 2022).

In some embodiments, spatial analysis can be performed using dedicated hardware and/or software, such as any of the systems described in Sections (II)(e)(ii) and/or (V) of PCT Publication No. WO2020/176788 and/or U.S. Patent Application Publication No. 2020/0277663, or any of one or more of the devices or methods described in Sections Control Slide for Imaging, Methods of Using Control Slides and Substrates for, Systems of Using Control Slides and Substrates for Imaging, and/or Sample and Array Alignment Devices and Methods, Informational labels of PCT Publication No. WO2020/123320.

Suitable systems for performing spatial analysis can include components such as a chamber (e.g., a flow cell or sealable, fluid-tight chamber) for containing a biological sample. The biological sample can be mounted for example, in a biological sample holder. One or more fluid chambers can be connected to the chamber and/or the sample holder via fluid conduits, and fluids can be delivered into the chamber and/or sample holder via fluidic pumps, vacuum sources, or other devices coupled to the fluid conduits that create a pressure gradient to drive fluid flow. One or more valves can also be connected to fluid conduits to regulate the flow of reagents from reservoirs to the chamber and/or sample holder.

The systems can optionally include a control unit that includes one or more electronic processors, an input interface, an output interface (such as a display), and a storage unit (e.g., a solid state storage medium such as, but not limited to, a magnetic, optical, or other solid state, persistent, writeable and/or re-writeable storage medium). The control unit can optionally be connected to one or more remote devices via a network. The control unit (and components thereof) can generally perform any of the steps and functions described herein. Where the system is connected to a remote device, the remote device (or devices) can perform any of the steps or features described herein. The systems can optionally include one or more detectors (e.g., CCD, CMOS) used to capture images. The systems can also optionally include one or more light sources (e.g., LED-based, diode-based, lasers) for illuminating a sample, a substrate with features, analytes from a biological sample captured on a substrate, and various control and calibration media.

The systems can optionally include software instructions encoded and/or implemented in one or more of tangible storage media and hardware components such as application specific integrated circuits. The software instructions, when executed by a control unit (and in particular, an electronic processor) or an integrated circuit, can cause the control unit, integrated circuit, or other component executing the software instructions to perform any of the method steps or functions described herein.

In some cases, the systems described herein can detect (e.g., register an image) the biological sample on the array. Exemplary methods to detect the biological sample on an array are described in PCT Publication No. WO2021/102003 and/or U.S. Patent Application Publication No. 2021/0150707, each of which is incorporated herein by reference in their entireties.

Prior to transferring analytes from the biological sample to the array of features on the substrate, the biological sample can be aligned with the array. Alignment of a biological sample and an array of features including capture probes can facilitate spatial analysis, which can be used to detect differences in analyte presence and/or level within different positions in the biological sample, for example, to generate a three-dimensional map of the analyte presence and/or level. Exemplary methods to generate a two- and/or three-dimensional map of the analyte presence and/or level are described in PCT Publication No. WO2020/053655 and spatial analysis methods are generally described in PCT Publication No. WO2021/102039 and/or U.S. Patent Application Publication No. 2021/0155982, each of which is incorporated herein by reference in their entireties.

In some cases, a map of analyte presence and/or level can be aligned to an image of a biological sample using one or more fiducial markers, e.g., objects placed in the field of view of an imaging system which appear in the image produced, as described in the Substrate Attributes Section, Control Slide for Imaging Section of PCT Publication Nos. WO2020/123320, WO 2021/102005, and/or U.S. Patent Application Publication No. 2021/0158522, each of which is incorporated herein by reference in their entireties. Fiducial markers can be used as a point of reference or measurement scale for alignment (e.g., to align a sample and an array, to align two substrates, to determine a location of a sample or array on a substrate relative to a fiducial marker) and/or for quantitative measurements of sizes and/or distances.

II. Detection of Nucleic Acids from Fixed Biological Samples

The methods described herein employ reverse transcription to generate a population of nucleic acids (e.g., a population of first strands or extended capture probes). The terms “first strand” or “extended capture probe” or “cDNA molecule” can be used interchangeably herein.

Second strand synthesis can be performed to generate a second strand by using a first strand as a template. In some instances, such methods can employ both a first primer comprising a sequence that is complementary to a TSO (or a portion thereof) that is appended to the target nucleic acid and copied via extension of the first strand and a second primer comprising a sequence of random nucleic acids. Such primers can be referred herein as a “TSO primer” or a “primer complementary to a TSO sequence” and a “primer with a random nucleotide sequence” or a “randomer primer”, respectively. By using a combination of such primers, both TSO-containing nucleotides and nucleotides lacking TSO regions can be detected.

Thus, in one non-limiting aspect, a method of determining a location of a nucleic acid in a biological sample includes: (a) contacting the nucleic acids from a biological sample with a substrate, wherein the substrate comprises a plurality of capture probes attached to the surface of the substrate, and wherein a capture probe of the plurality of capture probes comprises a spatial barcode (i.e., for determining the location of the captured nucleic acid in the biological sample) and a capture domain (that hybridizes to a sequence in the nucleic acids); (b) hybridizing the nucleic acid to the capture probe; (c) generating a population of extended capture probes by reverse transcription using the hybridized nucleic acid as a template, wherein a first extended capture probe in the population comprises a cDNA portion of the nucleic acid and a reverse complement of a TSO (rcTSO) that was appended to the hybridized nucleic acid, and wherein a second extended capture probe in the population comprises a cDNA portion of the nucleic acid, which may or may not, lack an rcTSO; (d) performing second strand synthesis in the presence of a composition comprising a first primer and/or a second primer, wherein the first primer comprises a sequence of a TSO which hybridizes to the rcTSO, and wherein the second primer comprises a random sequence of nucleotides; (e) performing second strand synthesis using the first primer and the second primer as primers from second strand synthesis, thereby generating a nucleic acid that comprises the target nucleic acid sequence, or a portion thereof, and sequences of the capture probe including the spatial barcode and other capture probe sequences, or complements thereof; (f) determining (i) all of the sequence of the spatial barcode or the complement thereof, and (ii) all or a portion of the sequence of the nucleic acid, or a complement thereof; and (g) using the determined sequences of (i) and (ii) to identify the location of the nucleic acid in the biological sample.

In another non-limiting aspect, a method of processing a nucleic acid from a biological sample includes: (a) hybridizing the nucleic acid from the biological sample to a capture probe, wherein the capture probe comprises a capture domain and a spatial barcode; (b) performing reverse transcription using the hybridized nucleic acid as a template in the presence of a TSO, thereby generating a population of cDNA molecules of the analyte, wherein a first cDNA molecule in the population comprises a rcTSO (i.e., a TSO was appended successfully to the hybridized nucleic acid), and wherein a second cDNA molecule in the population lacks a rcTSO (i.e., a TSO was unsuccessfully appended to the hybridized nucleic acid); and (c) performing second strand synthesis in the presence of a first primer and a second primer, wherein the first primer comprises a sequence that hybridizes to the rcTSO sequence, if it is present, and wherein the second primer is a randomer.

In some non-limiting instances, use of a TSO primer and a randomer primer can allow for detection of analytes without requiring a priori knowledge of the sequence of the nucleic acid. For example and without limitation, a TSO primer can be used in conjunction with methods that capture a common transcript sequence (e.g., a poly(A) sequence) and that prime second strand synthesis with template switching, and a randomer primer can be used to identify sequences that have not effectively incorporated a rcTSO into a first strand cDNA. Such a combination of primers could provide increased and enhanced sensitivity of spatial analysis methods when the captured nucleic acid is less amenable for labeling with TSO sequences, while preserving the ability to detect nucleic acids without using a sequence-specific probe (e.g., RTL methods as disclosed here).

Such primers can be used in the presence of captured nucleic acids that hybridize to a capture probe.

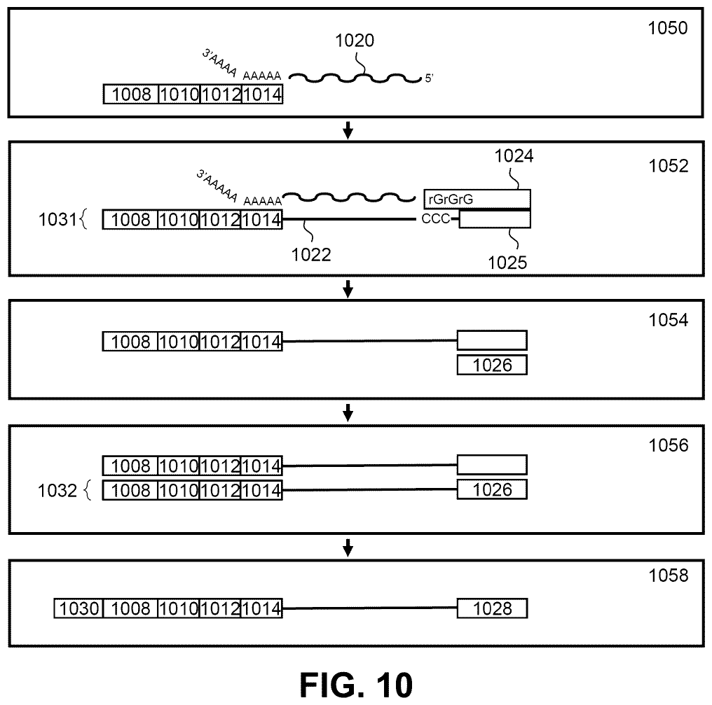

FIG. 10 shows a non-limiting, exemplary method for generating a second strand synthesis product with a TSO primer. In 1050, nucleic acid 1020 is hybridized to a capture probe via the capture probe domain 1014, which in this example is a poly(T) capture sequence hybridized to a poly(A) tail of an mRNA 1020. The mRNA as seen in this example has migrated from its location in a biological sample to a capture probe proximal to its location in the biological sample. In some instances, to facilitate the migration of a nucleic acid from a biological sample to a proximal capture probe on an arrayed substrate, the biological sample is processed in some manner, such as deparaffinization and decrosslinking of a FFPE sample, permeabilization of a biological sample, and the like. Tissue processing conditions are described below in Section II (b). The capture probe generally comprises several sequence domains: a functional domain 1008 such as a sequencing related domain, a spatial barcode domain 1010, a UMI domain 1012, and a capture domain 1014 (see FIG. 5 for additional information on capture probe domains).

In 1052, the capture domain 1014 can be extended by a reverse transcription reaction using the hybridized mRNA as a template to produce an extended capture probe (or first strand cDNA) 1031 having a cDNA portion 1022 complementary to a portion of the mRNA and also includes each of the sequence segments 1008, 1010, 1012, and 1014 of the capture probe. Terminal transferase activity of the reverse transcriptase adds additional bases to the cDNA portion (e.g., a poly(C) sequence). A TSO 1024 hybridizes to the poly(C) bases, and the cDNA is extended further to incorporate the complement of the TSO sequence (rcTSO) 1025 into the first strand cDNA 1031.

In some non-limiting instances, not all of the hybridized mRNA are labeled with a TSO. For example and without limitation, the captured mRNA can be degraded, in which addition of TSO is challenging or reduced. Thus, in some embodiments, a population of first strands generated during operation 1052 may include nucleic acids that may or may not lack an rcTSO 1025. In other embodiments, a population of first strands generated during operation 1052 can include a first population of nucleic acids having an rcTSO and a second population of nucleic acid lacking an rcTSO.

Reverse transcription can be performed in any useful manner. In some cases, reverse transcription includes extending the capture probe to generate a cDNA using the nucleic acid as a template, adding additional bases to the cDNA portion (e.g., a poly(C) sequence), and after hybridizing a TSO to the poly(C) sequence as seen in this example, further extending the cDNA portion to include an rcTSO formed by using the TSO as a template. To facilitate hybridizing the TSO to the homopolymer sequence (e.g., poly(C)), the TSO can be designed to include a sequence that is complementary to those additional bases. In some cases, the TSO can include a poly(G) sequence that is complementary to the poly(C) sequence. In particular cases, the poly(G) sequence includes RNA bases.

In some cases, the TSO includes DNA, RNA, or a combination thereof. In other cases, the TSO includes a homopolymer guanine sequence that hybridizes to a homopolymer cytosine sequence on the extended capture probe. In yet other cases, the TSO includes one or more non-natural (or modified) nucleic acids. In some cases, the rcTSO includes DNA.

The TSO can have any useful length. In some cases, the length of a TSO may be at least 5, at least 10, at least 15, at least 20, at least 30, at least 40, at least 50 nucleotides or longer.

As described herein, the TSO may or may not append to a nucleic acid or all the nucleic acids. Thus, in some cases, reverse transcription includes: (i) extending the capture probe using the TSO and the nucleic acid as a template, thereby generating a first extended capture probe including a cDNA portion and an rcTSO. The cDNA portion can include a sequence that is complementary to all or a portion of the hybridized nucleic acid. In some cases, reverse transcription includes: (ii) extending the capture probe using the hybridized nucleic acid as a template, wherein the nucleic acid is not in proximity to a TSO or the template lacks a TSO, thereby generating a second extended capture probe including a cDNA portion but not an rcTSO. In some cases, operations (i) and (ii) can occur in any order or simultaneously. In other cases, operation (i) occurs more frequently than operation (ii) during reverse transcription. In yet other cases, operation (i) occurs less frequently than operation (ii) during reverse transcription.

The step of extending the capture probe, e.g., by reverse transcription, may be performed using any suitable enzymes and protocols that many exist in the art. For instance, reverse transcription can be conducted in the presence of a reverse transcription enzyme comprising one or more of terminal transferase activity, template switching ability, strand displacement ability, or combinations thereof. In some instances, the reverse transcription enzyme comprises a Moloney Murine Leukemia Virus (M-MLV) reverse transcriptase enzyme (e.g., M-MLV reverse transcriptase enzyme 42B). Yet other non-limiting reverse transcriptase enzymes include Murine Leukemia Virus (MuLV), avian myeloblastosis virus (AMV), human immunodeficiency virus (HIV, e.g., HIV type 1), ArrayScript™, MultiScribe™, ThermoScript™, and SuperScript® I, II, III, and IV enzymes. A reverse transcriptase includes not only naturally occurring enzymes, but all such modified derivatives thereof, including also derivatives of naturally-occurring reverse transcriptase enzymes.

In addition, reverse transcription can be performed using sequence-modified derivatives or mutants of M-MLV, MuLV, AMV, and HIV reverse transcriptase enzymes, including mutants that retain at least some of the functional, e.g., reverse transcriptase, activity of the wild-type sequence. The reverse transcriptase enzyme can be provided as part of a composition that includes other components, e.g., stabilizing components that enhance or improve the activity of the reverse transcriptase enzyme, such as RNase inhibitor(s), inhibitors of DNA-dependent DNA synthesis, e.g., actinomycin D. Many sequence-modified derivative or mutants of reverse transcriptase enzymes, e.g., M-MLV, and compositions including unmodified and modified enzymes are commercially available, e.g., ArrayScript™ MultiScribe™, ThermoScript™, and SuperScript® I, II, III, and IV enzymes.

Certain reverse transcriptase enzymes (e.g., AMV reverse transcriptase and M-MLV reverse transcriptase) can synthesize a complementary DNA strand using both RNA (cDNA synthesis) and single-stranded DNA (ssDNA) as a template. Thus, in some embodiments, the reverse transcription reaction can use an enzyme (a reverse transcriptase) that is capable of using both RNA and ssDNA as the template for an extension reaction, e.g., an AMV or M-MLV reverse transcriptase.

Non-limiting reverse transcription reactions can employ a reaction mixture including a reverse transcriptase, dNTPs, and a suitable buffer. The reaction mixture may comprise other components, e.g. RNase inhibitor(s). Each dNTP can be present in any useful amount (e.g., ranging from about 10 to 5000 μM, usually from about 20 to 1000 μM). It will be evident that an equivalent reaction may be performed to generate a complementary strand of a captured DNA molecule, using an enzyme with DNA polymerase activity. Any useful conditions during reverse transcription can be employed, such as a temperature from about 20° C. to 45° C., or even higher with engineered or thermophilic reverse transcriptase (e.g., from about 53° C. to 75° C.), and for any useful period of time (e.g., from about 0.5, 1, 1.5, 2, or more hours).

Reverse transcription can include template switching. In one example of template switching, cDNA can be generated from reverse transcription of a template, e.g., any analyte described herein, where a reverse transcriptase with terminal transferase activity can add additional nucleotides, e.g., a poly(C) sequence, to the 3′ end of a cDNA molecule. The overhang may provide a target sequence to which a TSO can hybridize, thereby providing an additional template for further extension of the cDNA molecule. In particular cases, the TSO can contain an amplification domain sequence, the complement of which can be incorporated into the synthesized cDNA molecule.

In some embodiments, a TSO can include a sequence portion that is complementary to the additional nucleotides, e.g., the TSO can include a poly(G) sequence, preferably a ribo poly(G), or rGn, sequence. The additional nucleotides (e.g., a poly(C) sequence) on the cDNA can hybridize to the sequences complementary to the additional nucleotides (e.g., a ribo poly(G) sequence) on the TSO, whereby the TSO can be used by the reverse transcriptase as a template to further extend the cDNA molecule. TSOs may comprise deoxyribonucleic acids, ribonucleic acids, modified nucleic acids including locked nucleic acids (LNA), or any combination thereof.

Following operation 1052, the first strand 1031 can be primed with a TSO primer 1026 (e.g., a PCR primer complementary to the rcTSO region 1025) in operation 1054. The TSO primer 1026 may include a sequence of the entire sequence of the TSO 1024 or a portion of the sequence of the TSO 1054. For instance and without limitation, the TSO primer 1026 may lack the poly(G) sequence shown in the TSO 1024.

Second strand synthesis is shown in operation 1056 to provide a double stranded cDNA using the TSO primer 1026 and the cDNA portion 1022 as the template. The second strand 1032 can include the TSO primer 1026, a reverse complement of the cDNA portion 1022, and a reverse complement of different segments of the capture probe (a reverse complement of segments 1008, 1010, 1012, and 1014).

Release of the second strand synthesis product from the first strand cDNA can be conducted in any useful manner (e.g., physical denaturation, enzymatic reaction, chemical denaturation, or a combination thereof). Physical denaturation can include the use of heat to denature a double-stranded molecule. If amplification is carried out in situ on the array, this can encompass releasing amplicons by denaturation in the cycling reaction. As an alternative or addition to the use of a temperature sufficient to disrupt the hydrogen bonding, the solution may comprise salts, surfactants, alkaline agent, and the like, which may further destabilize the interaction between the nucleic acid molecules, resulting in the release of the second strands. In some instances, second strand synthesis can be conducted with a polymerase comprising strand displacement ability. In some instances, the polymerase is a Bst polymerase. Other enzymatic reactions can be employed. For example, enzymatic cleavage of a cleavage domain can destabilize a double-stranded structure, thereby resulting in release of the second strand.

The released second strands can be further processed, such as by fragmentation, end-repair, A-tailing, indexing, adaptor ligation, amplification, or other operations useful for library preparation. As seen in operation 1058, the extended product can be ligated to functional sequences. Optionally, prior to amplification (e.g., via PCR), the extended product can be cleaved from the array. In some non-limiting instances, the TSO primer 1026 can be removed. Functional sequences can be added to any end of the nucleic acid (e.g., see functional sequence 1028 and functional sequence 1030). Furthermore, functional sequences 1028, 1030 can represent one or more functional sequences described, e.g., a sample index sequence, a sequencer specific flow cell attachment sequence, and/or other functional sequences useful for specific priming, attachment, index, and other operational sequences used in systems, e.g., systems available from Illumina, Ion Torrent, Oxford Nanopore, Genia, Pacific Biosciences, Complete Genomics, and the like.

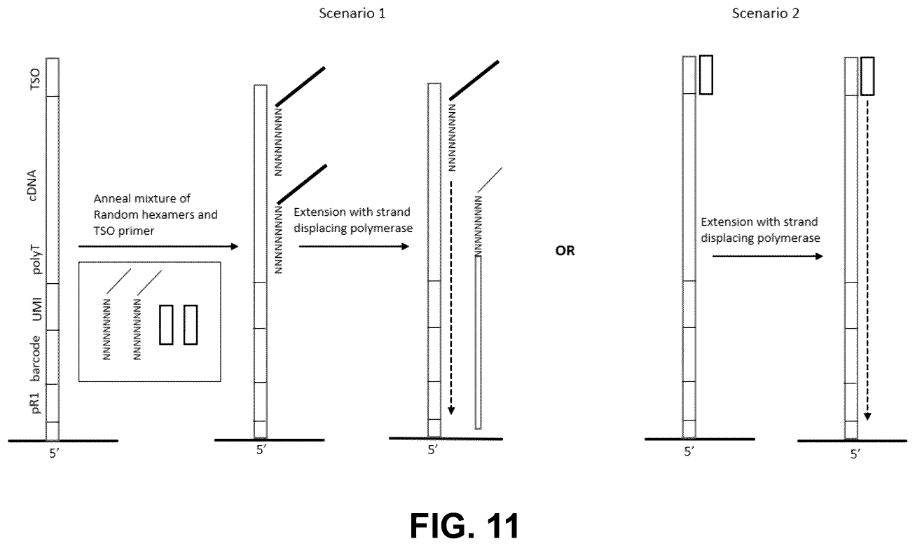

FIG. 11 provides a non-limiting example of a combination of second strand synthesis events. Generated second strands can be generated by using a TSO primer (Scenario 2) or a randomer primer (Scenario 1). When a TSO primer anneals to the rcTSO region of the first strand (if TSO addition is successful), then the synthesized second strand can extend along the length of the first strand and incorporate the sequences of the capture probe, including the spatial barcode and other functional regions that are present in the capture probe as shown in Scenario 2. However, for those instances where a TSO is not appended to the hybridized nucleic acid and thus a rcTSO is not generated by continued extension of the first strand synthesis, a randomer primer can be used which hybridizes at random positions along the first strand. While the resultant second strand (a randomer-primed strand) will be shorter, and wherein multiples of second strands of differential lengths based on the hybridizing of the randomer primer on the first strand cDNA may be generated, the second strand products based on the randomer primer extension will still include the sequences of the capture probe, including the spatial barcode and the functional sequences as found in the capture probe.

In some embodiments, once a cDNA molecule is generated, the TSO primer can be employed in a cDNA amplification reaction (e.g., with a DNA polymerase). In some embodiments, double stranded cDNA (e.g., first strand cDNA and second strand reverse complement cDNA) can be amplified via isothermal amplification with either a helicase or recombinase, followed by a strand displacing DNA polymerase. The strand displacing DNA polymerase can generate a displaced second strand resulting in an amplified product.

Second strand synthesis can be performed in any useful manner. In some cases, second strand synthesis includes: (i) hybridizing a TSO primer to a rcTSO of a first strand (or an extended capture probe); and (ii) extending the TSO primer using the extended capture probe as a template, thereby generating a second strand, wherein the second strand is complementary to all or a portion of the analyte and all of the capture probe. In other cases, second strand synthesis includes: (i) hybridizing a randomer primer to a random position (e.g., within a cDNA portion) of a first strand (or an extended capture probe); and (ii) extending the randomer primer using the first strand as a template, thereby generating a second strand, wherein the second strand is complementary to all or a portion of the analyte and all of the capture probe.

Any useful conditions during second strand synthesis can be employed. In some embodiments, second strand synthesis can be performed on the substrate by subjecting the substrate to a thermocycling protocol (e.g., pre-equilibrate at 65° C., second strand synthesis at 65° C. for about 10 to 15 minutes, then hold at 4° C. for 10 to 30 minutes, and incubate at 30° C. for 30 minutes to 2 hours).

Second strand synthesis may occur in situ on the array, which in turn provides an attachment surface for a first strand. Alternatively, the first strand may be released from the array, and then second strand synthesis can be performed in the presence of the TSO primer and/or the randomer primer. Optionally, second strand synthesis may be followed by amplification to increase the amount of cDNA molecules to yield quantities that are sufficient for sequencing. In some cases, second strand synthesis can include incorporation of an amplification domain to the cDNA molecule (e.g., by use of primers including such a domain, by ligation, by template switching, or by terminal transferase modification), in which the amplification domain comprises a distinct sequence to which an amplification primer may hybridize.

### (a) TSO Primers and Randomer Primers

In some embodiments, a TSO primer is present, and the TSO primer can include any sequence that binds to or hybridized with the rcTSO region of the first strand. In some cases, the TSO primer includes a sequence of a TSO (a TSO sequence) or a sequence having substantial identity to the TSO sequence (e.g., a sequence that has at least about 80%, 85%, 90%, 95%, 96%, 97%, 98%, 99%, or 100% sequence identity to the reference nucleic acid sequence).

The TSO primer can have any useful length (e.g., from about 10 to 50 nucleotides in length. In some cases, the length of a TSO primer may be 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 nucleotides or longer.

The TSO primer may or may not be of the same length as the TSO that is added to the hybridized nucleic acid and provided during template switching. In some cases, the TSO primer may be shorter than the TSO added during template switching. For instance, during template switching, the TSO may include a first region (e.g., a poly(G) sequence) that hybridizes to an untemplated overhang (e.g., a poly(C) sequence) added to cDNA portion of the extended capture probe. In some cases, the TSO primer may include a sequence that is substantially similar to the sequence of the TSO, and the TSO primer may further lack that first region. In other cases, the TSO primer is shorter than the TSO (e.g., shorter by 1, 2, 3, 4, 5, 6, 7, 8, 9, 10, 11, 12, 13, 14, 15, 16, 17, 18, 19, 20 or more nucleotides). In some cases, the TSO primer lacks the poly(G) sequence that may be present in the TSO. In some cases, the TSO primer is a fragment lacking a portion of a sequence located at the 3′ end of the TSO. In particular cases, the fragment can be at least about 10%, 20%, 30%, 40%, 50%, 60%, 70%, 80%, or 90% of the entire length of the TSO.

In some cases, the TSO primer includes DNA. In some cases, the TSO includes a sequence that hybridizes to the capture probe or the extended capture probe (e.g., a 3′ or 5′ end of the capture probe or extended capture probe).

As described herein, randomer primers are employed to recover first strands lacking a TSO region. In use, a randomer primer can hybridize to both types of first strand cDNA molecules that may include a TSO or may lack a TSO. Typically, the randomer primer would hybridize at a random position, i.e. within the sequence rather than at the end of the sequence, however the randomer primer may hybridize at the end of a first strand. Randomer primers are designed to hybridize randomly across all or a portion in the first strand cDNA product.

Randomer primers of varying lengths can be optimized in order to find ideal melting temperatures (Tₘ) during hybridization of the randomer primer to the first strand (e.g., a random position within a cDNA). In some instances, the randomer primer can include a domain that is not a random sequence, but instead comprises one or more other functional domains, such as a unique molecular identifier, a universal sequence, a cleavage domain, and combinations thereof. In specific instances, the randomer primer comprises a hexamer (e.g., a six-nucleotide sequence). In other specific instances, the randomer primer comprises a nonomer (e.g., a nine-nucleotide sequence). In some instances, the randomer primers include a random sequence of at least 4 to about 16 nucleotides in length (e.g., 4, 5, 6, 7, 8, 9, 10, 11, 12, 13, 14, 15, or 16).

In some instances, a universal sequence can be a primer sequence such as a sequencing primer sequence or an amplification primer sequence, thereby allowing for extended sequences having the handle to generate amplicons or to be captured by a complementary sequence, such as a sequencing primer sequence.

In some embodiments, the randomer primer comprises at least one non-natural nucleic acid in its sequence. In some embodiments, the non-natural nucleic acid is a locked nucleic acid (LNA). In some embodiments, the randomer primer comprises one or more modifications to its structure. In other embodiments, the randomer primer includes natural nucleic acids (e.g., mostly or all natural nucleic acids).

### (b) Processing of Biological Samples

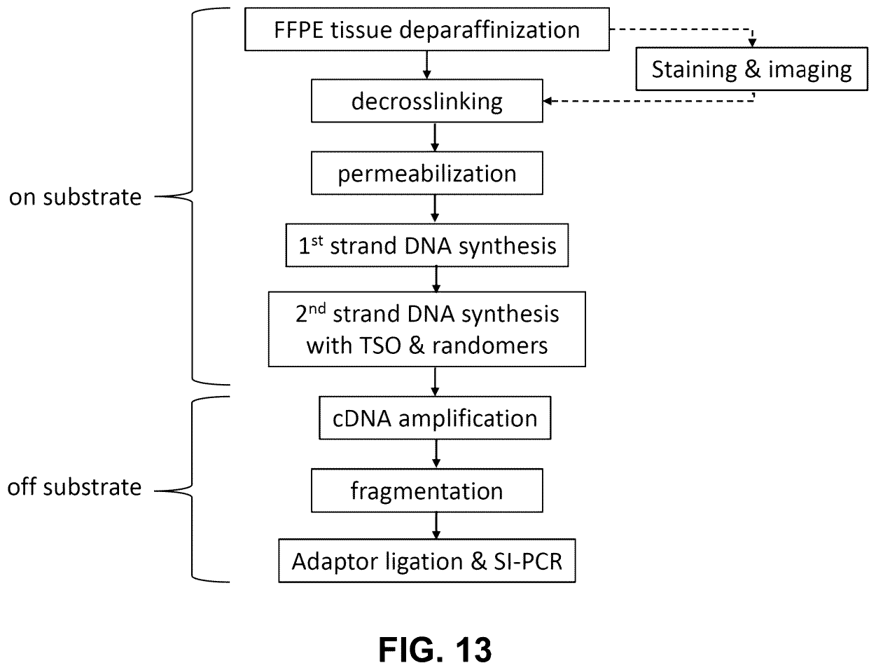

A biological sample can be processed in any useful manner, and certain processed samples may benefit from use with the methods herein. In particular embodiments, processing can include those useful for preparing FFPE samples that would benefit from the reverse transcription and second strand synthesis methods described herein (e.g., including template switching and priming with TSO primers and/or randomer primers). FIG. 13 shows a non-limiting workflow that includes processing operations (e.g., deparaffinization, imaging, decrosslinking, permeabilization, and the like).

As described herein, the present disclosure encompasses methods that may be suitable for use with biological samples that may be degraded (e.g., biological samples that have been fixed, embedded in paraffin, and/or frozen). In some cases, the biological sample can be fixed with methanol, paraformaldehyde, formaldehyde, or other fixatives (e.g., any described herein). In some cases, the biological sample is an FFPE sample. Following fixation of the tissue sample and embedding in a paraffin or resin block, the tissue samples may sectioned, i.e. thinly sliced, onto the array. Other fixatives and/or embedding materials can be used.

Prior to use, the biological sample may be processed to remove embedding material e.g., to deparaffinize, i.e. to remove the paraffin or resin, from the sample. This may be achieved by any suitable method and the removal of paraffin or resin or other material from tissue samples is well established in the art, e.g. by incubating the sample (on the surface of the array) in an appropriate solvent e.g. xylene, e.g. twice for 10 minutes, followed by an ethanol rinse, e.g. 99.5% ethanol for 2 minutes, 96% ethanol for 2 minutes, and 70% ethanol for 2 minutes.

It will be evident to the skilled person that the RNA in tissue sections prepared using methods of FFPE or other methods of fixing and embedding is more likely to be partially degraded than in the case of frozen tissue. However, without wishing to be bound by any particular theory, it is believed that this may be addressed by the methods described herein. For instance, if the RNA in the sample is partially degraded, then addition of a TSO to degraded RNA may be less efficient (as compared to non-degraded RNA). Thus, the use of TSO primers and randomer primers may detect both TSO-labeled and non-TSO-labeled nucleic acids.

After a fixed (e.g., FFPE, PFA, acetone, methanol) biological sample has undergone deparaffinization, the fixed (e.g., FFPE, PFA) biological sample can be further processed. For example, fixed (e.g., FFPE, PFA) biological samples can be treated to remove crosslinks (e.g., decrosslinking). In some embodiments, decrosslinking the crosslinks (e.g., formaldehyde-induced crosslinks) in the fixed (e.g., FFPE, PFA) biological sample can include treating the sample with heat or a decrosslinking agent. In some embodiments, decrosslinking can include performing a chemical reaction (e.g., with a decrosslinking agent). In some embodiments, decrosslinking can include heat, a chemical reaction, and/or permeabilization agents.

In some embodiments, decrosslinking can be performed in the presence of a buffer. In some embodiments, the buffer is Tris-EDTA (TE) buffer (e.g., TE buffer for FFPE biological samples). In some embodiments, the buffer is citrate buffer (e.g., citrate buffer for FFPE biological samples). In some embodiments, the buffer is Tris-HCl buffer (e.g., Tris-HCl buffer for PFA fixed biological samples). In some embodiments, the buffer (e.g., TE buffer, Tris-HCl buffer) has a pH of about 5.0 to about 10.0 and a temperature between about 60° C. to 100° C. (e.g., about 60° C., 65° C., 70° C., 75° C., 80° C., 85° C., 90° C., 95° C., or 100° C.) for a period of about 10 minutes to 3 hours (e.g., about 10 minutes, 30 minutes, 1 hour, 1.5 hours, 2 hours, 2.5 hours, or 3 hours).

Any of the methods described herein or known in the art can be used to stain and/or image the biological sample. For example, in some embodiments, a biological sample is stained (e.g., via eosin and/or hematoxylin), imaged, destained (e.g., via HCl), or a combination thereof. Staining can occur after deparaffinization; and/or destaining can occur before or after decrosslinking. Destaining can occur with HCl (e.g., 0.1 N) at a temperature between about 30° C. to 50° C. (e.g., about 30° C., 32° C., 34° C., 36° C., 38° C., 40° C., 42° C., 44° C., 46° C., 48° C., or 50° C.) for a period of about 5 to 30 minutes (e.g., about 5, 10, 15, 20, 25, or 30 minutes).

In some cases, a biological sample can be treated with an RNase inactivating agent to neutralize exonucleases (e.g., an RNase inhibitor, a ribonucleoside vanadyl complex, EDTA, etc.). In some embodiments, the biological sample is treated with an RNase inactivating agent before hybridizing the nucleic acid to the capture probe. In some embodiments, the biological sample is treated with an RNase inactivating agent at the same time as hybridizing the analyte to the capture probe. In some embodiments, the biological sample is treated with an RNase inactivating agent after hybridizing the analyte to the capture probe. In some embodiments, hybridizing the analyte to the capture probe further comprises treating the biological sample with an RNase inactivating agent.

In some embodiments, the biological sample is permeabilized (e.g., permeabilized by any of the methods described herein). In some embodiments, the permeabilization is an enzymatic permeabilization. In some embodiments, the permeabilization is a chemical permeabilization. In some embodiments, the biological sample is permeabilized before hybridizing the nucleic acid to the capture probe. In some embodiments, the biological sample is permeabilized at the same time as hybridizing the nucleic acid to the capture probe.

In some embodiments, the biological sample is permeabilized from about 30 to about 120 minutes, from about 40 to about 110 minutes, from about 50 to about 100 minutes, from about 60 to about 90 minutes, or from about 70 to 80 minutes. In some embodiments, the biological samples is permeabilized about 30, about 35, about 40, about 45, about 50, about 55, about 60, about 65, about 70, about 75, about 80, about 90, about 95, about 100, about 105, about 110, about 115, or about 130 minutes.

In some cases, the permeabilization buffer can include one or more permeabilization agents. In some embodiments, the permeabilization buffer comprises urea. In some embodiments, the urea is at a concentration of about 0.5M to 3.0 M. In some embodiments, the concentration of the urea is about 0.5, 1.0, 1.5, 2.0, 2.5, or about 3.0 M. In some cases, the permeabilization buffer comprises an endopeptidase. In some cases, the endopeptidase is proteinase K. In some cases, the proteinase K is at a concentration of about 1 mg/mL to 5 mg/mL. In some cases, the concentration of the proteinase K is about 1, 1.5, 2, 2.5, 3, 3.5, 4, 4.5, or 5 mg/mL. In some embodiments, the permeabilization buffer comprises polyethylene glycol (PEG). In some embodiments, the PEG is from about PEG 2 K to about PEG 30 K. In some embodiments, the PEG is PEG 2 K, 3 K, 4 K, 5 K, 6 K, 7 K, 8 K, 9 K, 10 K, 11 K, 12 K, 13 K, 14 K, 15 K, 16 K, 17 K, 18 K, 19 K, 20 K, 21 K, 22 K, 23 K, 24 K, 25 K, 26 K, 27 K, 28 K, 29 K, or 30K. In some embodiments, the PEG is present at a concentration from about 2% to 25%, from about 4% to about 23%, from about 6% to about 21%, or from about 8% to about 20% (v/v). In some embodiments, the permeabilization buffer includes a detergent. In some embodiments, the detergent is sarkosyl. In some embodiments, the sarkosyl is present at about 2% to about 10% (v/v). In some embodiments, the sarkosyl is present at about 3%, 4%, 5%, 6%, 7%, 8%, or 9% (v/v).

### III. Kits

In some embodiments, also provided herein are kits that include one or more reagents to practice the methods as described herein. In some instances, the kit includes a substrate comprising a plurality of capture probes comprising a spatial barcode and a capture domain. In some embodiments, the kit includes one or more reagents selected from a buffer, a plurality of dNTPs, a plurality of TSOs, a plurality of sequences complementary to the TSOs or complementary to a portion of the TSOs, and a plurality of randomer sequences. In some embodiments the randomer sequence further comprise a sequence domain that is not random, but instead includes one or more functional sequences, universal sequences, cleavage sites, or a combination thereof. In some embodiments, the plurality of sequences complementary to the TSOs can include any TSO primer described herein. In some embodiments, the plurality of randomer sequences can include any randomer primer described herein. In some embodiments, the kit further includes one or more enzymes selected from a reverse transcriptase, a polymerase, or any enzyme described herein. In some embodiments, a kit can further include instructions for performing any of the methods or steps provided herein.

### EXAMPLES

### Example 1. Comparison of Two Spatial Chemistries in Assaying RNA from FFPE Samples in Different Tissue Types

Experiments were conducted to compare two methods for analyzing mRNA in fixed tissue samples. The first method is an RTL method as previously described (FIG. 9A and FIG. 9B), a method that was developed to spatially analyze target mRNA from FFPE samples, samples which are notorious for containing degraded mRNA in various degrees. The RTL method in this comparison serves as the gold standard due to its exceptional ability to capture and analyze mRNA from fixed tissue samples. The second method is being compared to the gold standard, the second method being the combination method developed and described herein where the mRNA in a fixed sample is directly hybridized to the capture probe and two different probe sets are used for second strand synthesis, in opposition to the RTL method where the ligation product is captured by the capture probe, the ligation product being a proxy of the target mRNA as such an indirect method of target mRNA analysis.

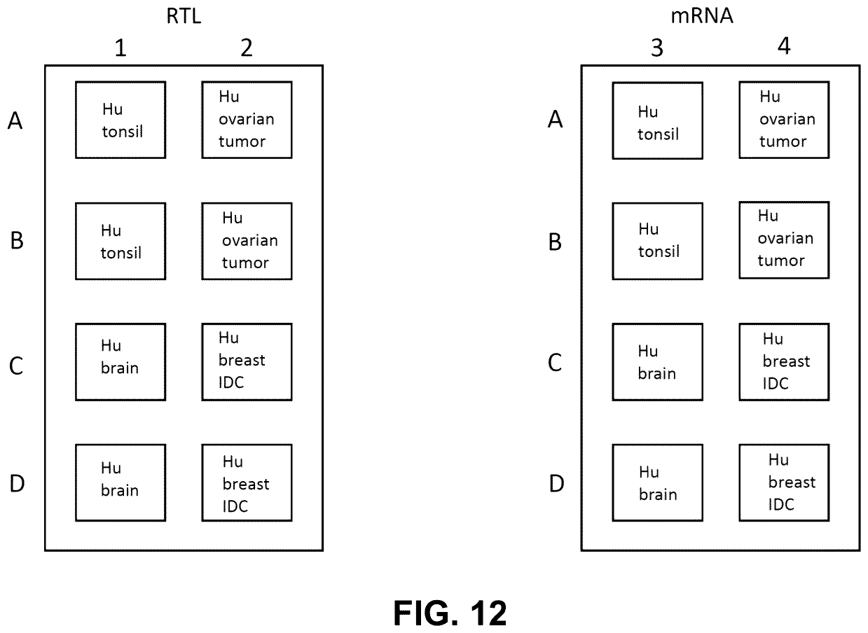

The following FFPE tissue samples were used in the experiments, in duplicate: human tonsil; human brain (the percentage of fragments of >200 nucleotides (DV200) of 27%); human ovarian cancer; and human breast (DV200 of 63.54%). FIG. 12 shows an exemplary experimental setup for the samples. There were two spatial arrayed slides, one for RTL method (RTL) and the second following the combination second strand synthesis method described herein for mRNA direct capture (mRNA). On each arrayed slide, there are eight separate spatially barcoded arrays and the location of the tissue samples are identified by column and row.

The following protocol was used on the FFPE tissues to allow access for the two primer sets to the nucleic acids in the tissues. The FFPE tissue sections were deparaffinized using xylenes, H & E stained, and imaged. Following the imaging, the tissues were destained with 0.1N HCl at 42° C. for 15 minutes. The tissue samples were decrosslinked by incubating the tissues in a citrate buffer at 95° C. for 1 hour, followed by permeabilization in a Proteinase K permeabilization solution for 1 hour. After permeabilization, the nucleic acids were allowed to migrate to the array surface for hybridization with the capture domain of the capture probes on the surface of the array. Reverse transcription was performed using a reverse transcriptase with a TSO at 42° C. for 1 hour followed by second strand synthesis in the presence of TSO primers and a randomer primers at 65° C. for 10 minutes, 4° C. for 20 minutes (slow ramp), and 30° C. for 1 hour. The second strand synthesis products were removed from the slide by the addition of KOH and placed in clean tubes for subsequent library preparation in anticipation of sequencing. Additionally, fragmentation was conducted for 2.5 minutes to accommodate short cDNA, but this step can be omitted, such that the protocol can proceed directly to library preparation. FIG. 13 shows an exemplary workflow for this protocol.

For the RTL comparative spatial assay, the protocol is described in the User Guide for Visium Spatial Gene Expression Reagent Kits for FFPE (CG000407), except that the destaining and decrosslinking were performed as above.

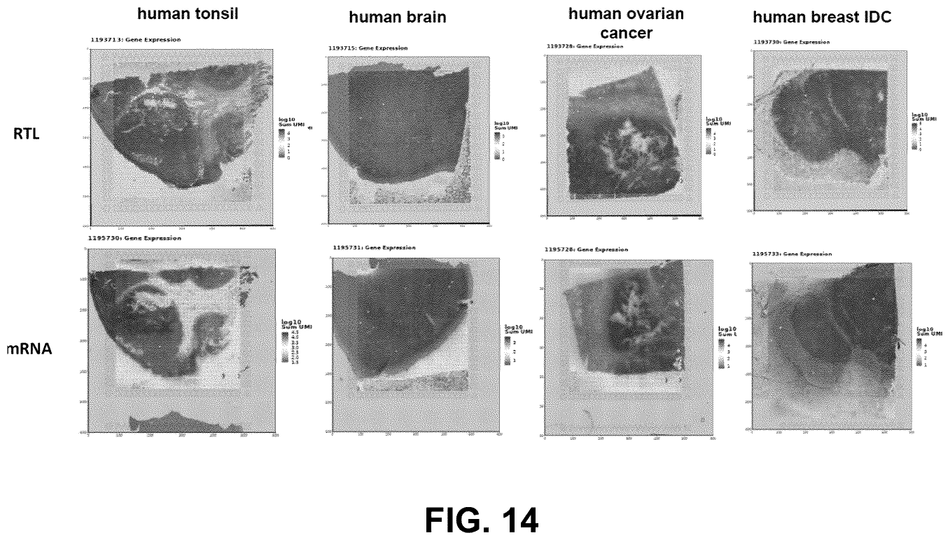

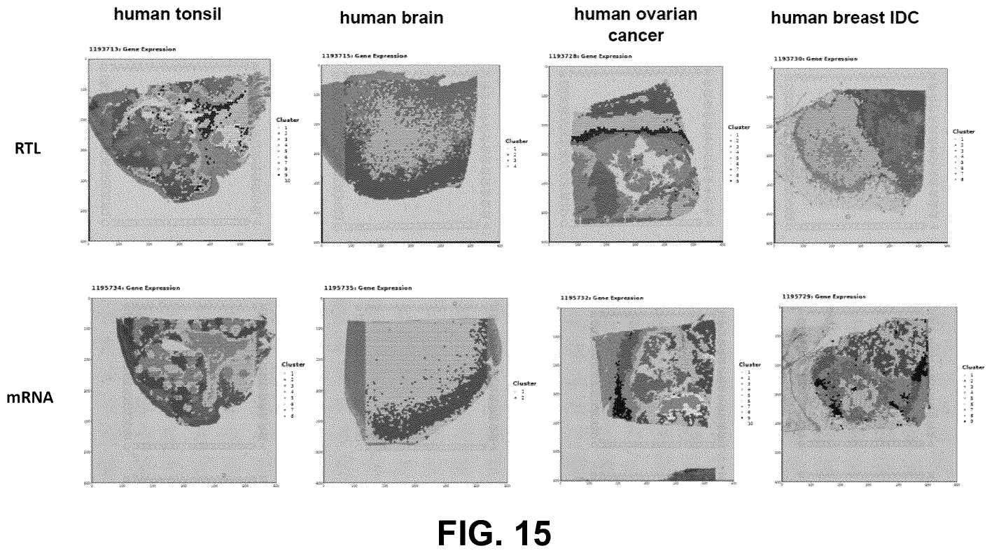

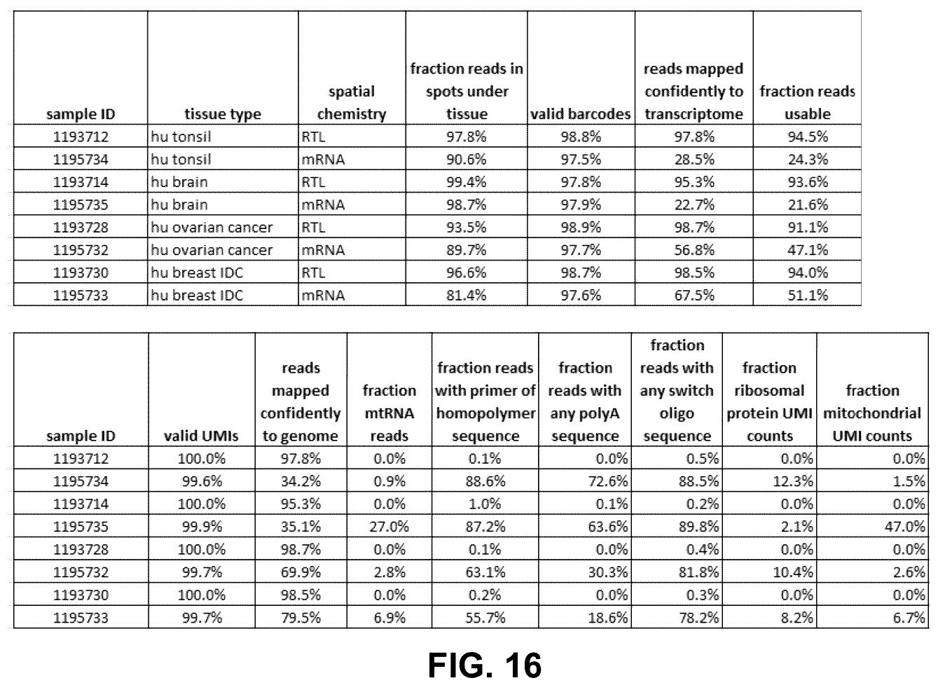

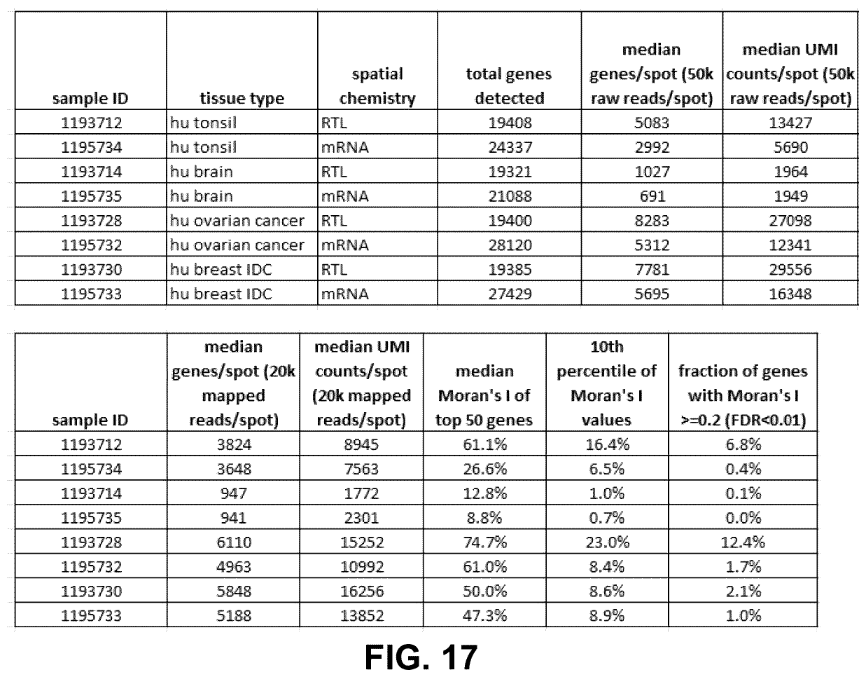

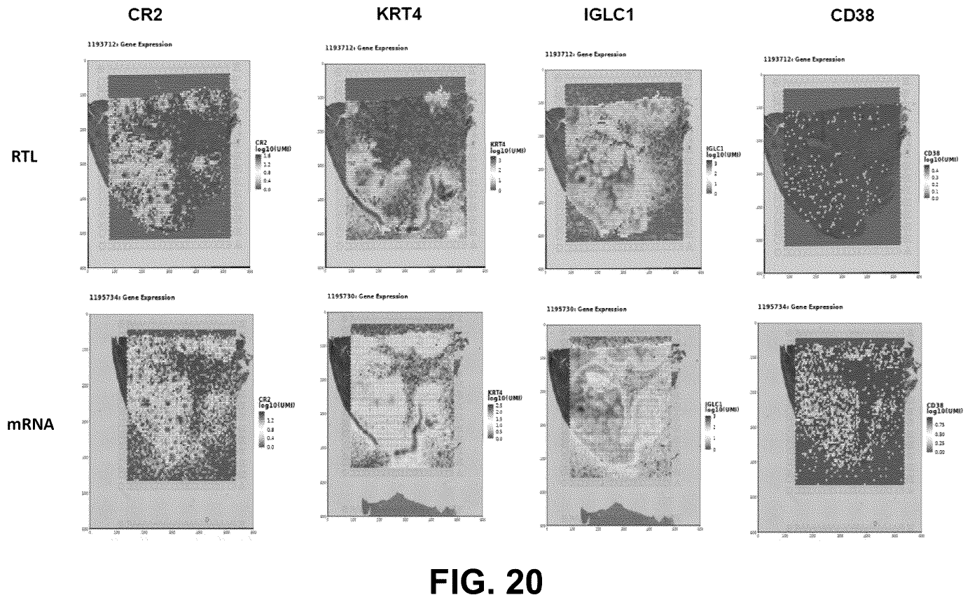

Spatial parameters were compared between the RTL method and the two primer combination method. UMI plots and clustering data are provided in FIG. 14 and FIG. 15, respectively. The UMI plots and clustering data were applied to each tissue section for each condition showing correlation between the spatial data regardless of method used. At an individual gene level of expression, FIG. 20 shows that, at least for human tonsil, gene expression of the chosen genes was comparable. Raw data for library quality and sensitivity are provided in FIG. 16 and FIG. 17, respectively.

As seen in FIG. 16, lower percentage of reads mapped back to the transcriptome or the genome for the two primer method data (mRNA). One potential reason for the lower mapping of sequencing reads could be that, for that sample method, there was an increased number of sequencing reads related to a homopolymer sequence (a polyA sequence), a TSO sequence, and mitochondrial counts. This effect was observed to be more pronounced in low quality samples (e.g., human tonsil sample ID 1195734 and human brain sample ID 1195735). In higher quality samples (e.g., human ovarian tumor sample ID 1195732, human breast IDC sample ID 1195733), the library quality was much higher, and typical of the normal mRNA capture RT/TSO workflow that is used on non-fixed tissue samples (typically around 70% for human tissues). Mitochondrial UMI counts were particularly high (47%) for samples with low DV200 (human brain sample ID 1195735).

As seen in FIG. 17, more total genes were detected using the two primer method (mRNA) regardless of sample type. This was not surprising, as the methodology for the RTL probes, as previously described, relies on mRNA sequences being known and probes being designed specifically to those sequences, converse to the capture design of the direct mRNA capture method. However, sensitivity was significantly higher using the RTL method at matched raw reads per spot due to low mapping for the mRNA method, as previously described. Sensitivity was similar when comparing mapped reads per spot, suggesting that a similar amount of genes is detected with the two primer combination method, however higher sequencing depth may be useful to account for a larger amount of wasted reads.

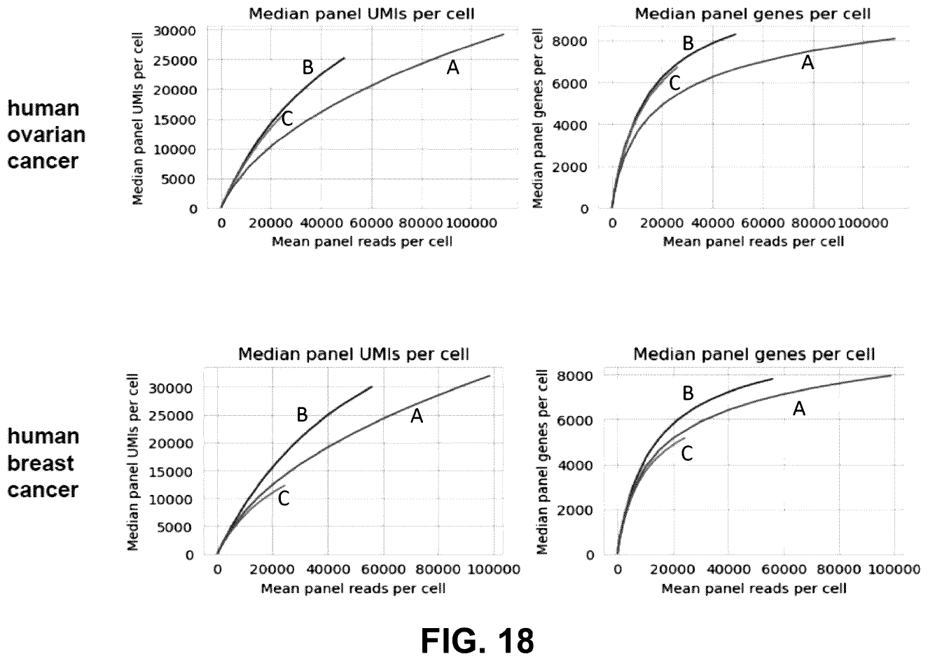

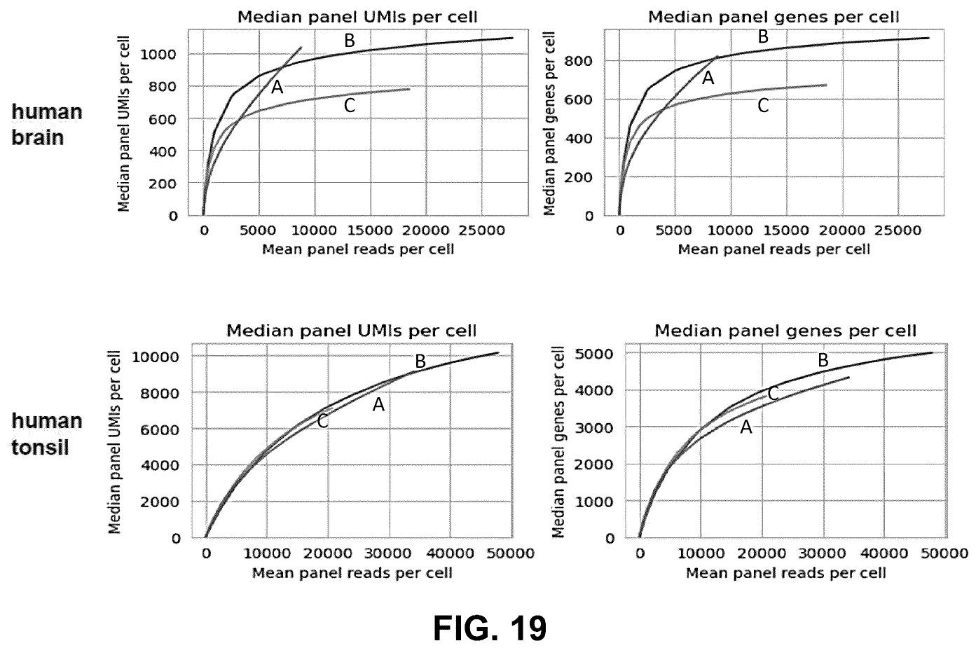

Saturation curves demonstrating the measure of the fraction of the library that was sequenced from a tissue are provided in FIG. 18 and FIG. 19. Sequencing saturation is dependent on the complexity of the library (Median Panel UMIs/cell) versus the sequencing depth (Mean Panel Reads/Cell). Different cell types will have different amounts of RNA and therefore will differ in the total number of transcript sequences that are represented in the library to be sequenced. Further, the deeper a library is sequenced, the greater the possibility of detecting more transcript sequences. The deeper sequencing however reaches saturation at different sequencing depths dependent on the cell type and library complexity, as previously described. FIG. 18 shows a replicate of the RTL method (B and C) with the two primer combination method being A. While the replicate C was not taken over a sequencing depth of around 30,000, it shows a similar trend to RTL B. The two primer method A is comparable to the RTL method in saturation curves for both the human breast and human ovarian cell types. That trend is closer still for human brain and human tonsil cell types seen in FIG. 19.

Overall, the combination of second strand synthesis scenarios 1 and 2 is successful in assaying nucleic acids, for example mRNA from FFPE tissue samples, with similar metrics observed as compared to the RTL FFPE method. The discovery of this new workflow combining direct capture of nucleic acids from a fixed biological sample, nucleic acids which may likely be degraded to some degree due to the fixation process, with a TSO primer and randomer primers for performing second strand synthesis and generating products which can be analyzed in downstream applications to correlate the location of the nucleic acids in the biological sample, provides an additional tool for spatial transcriptomics research.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method of generating a plurality of second strand synthesis products from a plurality of nucleic acids in a fixed biological sample, the method comprising:
(a) contacting a fixed biological sample with a substrate, wherein the substrate comprises a plurality of capture probes, and wherein a capture probe of the plurality of capture probes comprises a spatial barcode and a capture domain;
(b) hybridizing the plurality of nucleic acids to the plurality of capture probes;
(c) generating a population of extended capture probes by reverse transcription comprising:
(i) a first population of extended capture probes comprising a cDNA sequence of the hybridized nucleic acid and a TSO sequence, or a complement thereof, and
(ii) a second population of extended capture probes comprising a cDNA sequence of the nucleic acid and lacking a TSO sequence, or a complement thereof; and
(d) performing second strand synthesis on the first and second populations of extended capture probes in the presence of a plurality of first primers and a plurality of second primers, wherein the plurality of first primers comprise complementary TSO primers to hybridize to all or a portion of the TSO sequences, and wherein the plurality of second primers comprise randomers, thereby generating a plurality of second strand synthesis products from a plurality of nucleic acids in a fixed biological sample.

2. The method of claim 1, wherein the reverse transcription comprises:
(i) extending the capture probe using the hybridized nucleic acid as a template, and appending a polynucleotide sequence to the end of the extended capture probe;
(ii) hybridizing a TSO comprising a polyribonucleotide sequence complementary to the polynucleotide sequence, and extending further the extended capture probe to include the complementary sequences of the TSO, thereby generating the first population of extended capture probes comprising the cDNA sequences and the complementary TSO sequences; and
(iii) extending the capture probes using the hybridized nucleic acids as a template, thereby generating the second population of extended capture probes comprising the cDNA sequences.

3. The method of claim 1, wherein the second strand synthesis comprises:
(i) hybridizing the complementary TSO primers to the TSO sequence on the cDNA of the first population of extended capture probes and extending the TSO primer using the cDNA as a template; and
(ii) hybridizing the randomers to one or both of the first population of extended capture probes and the second population of extended capture probes, and extending the randomers using the cDNA as a template,
thereby generating second strand synthesis products comprising the cDNA sequences, or complements thereof, and the capture probe sequences, or complements thereof.

4. The method of claim 1, wherein the plurality of second primers comprises a defined sequence at the 5′ end of the randomer.

5. The method of claim 4, wherein the defined sequence is one or more of a universal sequence and a cleavage sequence, and wherein the universal sequence is a primer sequence or a sequence compatible with a sequencing platform, or both.

6. The method of claim 5, wherein the primer sequence is an amplification primer sequence.

7. A method of processing a nucleic acid from a biological sample, comprising:
(a) hybridizing the nucleic acid from the biological sample to a capture probe, wherein the capture probe comprises a capture domain and a spatial barcode;
(b) performing reverse transcription using the hybridized nucleic acid as a template in the presence of a template switching oligonucleotide (TSO), thereby generating a population of complementary DNA (cDNA) molecules of the analyte, wherein a first cDNA molecule in the population comprises a reverse complement of a template switching oligonucleotide (rcTSO), and wherein a second cDNA molecule in the population lacks a rcTSO; and
(c) performing second strand synthesis in the presence of a first primer and a second primer, wherein the first primer comprises a sequence that hybridizes to the rcTSO sequence, if it is present, and wherein the second primer is a randomer.

8. The method of claim 7, wherein the reverse transcription comprises:
(i) providing the TSO in proximity to a 5′ end of the analyte; and
(ii) extending the capture probe using the TSO and the analyte as a template, thereby generating the first cDNA molecule comprising the rcTSO and a sequence that is complementary to all or a portion of the analyte.

9. The method of claim 7, wherein the reverse transcription comprises:
(i) extending the capture probe using the analyte as a template, wherein the template lacks a TSO, thereby generating the second cDNA molecule comprising a sequence that is complementary to all or a portion of the analyte.

10. The method of claim 7, wherein the second strand synthesis comprises:
(i) hybridizing the TSO sequence of the first primer to the rcTSO of the first cDNA molecule; and
(ii) extending the TSO sequence of the first primer using the extended capture probe as a template, thereby generating a second strand, wherein the second strand is complementary to all or a portion of the analyte and all or a portion of the capture probe.

11. The method of claim 9, wherein the second strand synthesis comprises:
(i) hybridizing the randomer to the first cDNA molecule or the second cDNA molecule; and
(ii) extending the randomer using the extended capture probe as a template, thereby generating a second strand, wherein the second strand is complementary to all or a portion of the analyte and all or a portion of the capture probe.

12. The method of claim 1, wherein the capture probe further comprises one or more functional domains, a unique molecular identifier (UMI), a cleavage domain, and combinations thereof.

13. The method of claim 1, wherein said reverse transcription is conducted in the presence of a reverse transcription enzyme comprising one or more of terminal transferase activity, template switching ability, strand displacement ability, or combinations thereof.

14. The method of claim 13, wherein the reverse transcription enzyme comprises a Moloney Murine Leukemia Virus (M-MLV) reverse transcriptase enzyme or M-MLV reverse transcriptase enzyme 42B.

15. The method of claim 1, wherein the TSO is about 10 to 50 nucleotides in length.

16. The method of claim 1, wherein the TSO comprises DNA and/or RNA; and/or wherein the rcTSO comprises DNA.

17. The method of claim 1, wherein the TSO comprises a homopolymer guanine sequence that hybridizes to a homopolymer cytosine sequence on the capture probe or the extended capture probe.

18. The method of claim 1, wherein the randomer is about 4 to 16 nucleotides in length.

19. The method of claim 1, wherein second strand synthesis is performed concurrent with or immediately after first strand cDNA synthesis.

20. The method of claim 1, further comprising releasing the second strand synthesis products, wherein the releasing the second strand synthesis products comprises physical denaturation, enzymatic reaction, or chemical denaturation.

21. The method of claim 1, further comprising permeabilizing the biological sample with a permeabilization agent.

22. The method of claim 1, wherein the fixed biological sample is fixed with methanol, acetone, paraformaldehyde (PFA), formaldehyde, or a combination thereof.

23. The method of claim 1, wherein the fixed biological sample comprises a tissue section or a formalin-fixed, paraffin-embedded (FFPE) sample; and optionally wherein the FFPE tissue sample is deparaffinized or optionally wherein the FFPE tissue sample is decrosslinked with a decrosslinking agent.

24. The method of claim 1, wherein the nucleic acid is an RNA molecule or an mRNA molecule.

25. The method of claim 1, wherein the nucleic acid comprises a single nucleotide polymorphism (SNP).

26. The method of claim 1, wherein the nucleic acid is of non-human origin or non-mouse origin.

27. The method of claim 1, further comprises determining (i) all of the sequence of the spatial barcode of the extended capture probes and (ii) the sequence of all or a portion of the nucleic acid, and using (i) and (ii) to identify the location of the nucleic acid in the fixed biological sample.

28. The method of claim 27, wherein the determining comprises sequencing, and optionally wherein the sequencing comprises in situ sequencing, Sanger sequencing methods, next-generation sequencing methods, or nanopore sequencing.

29. A kit comprising:
(a) a substrate comprising a plurality of captures probes attached to the surface of the substrate, wherein a capture probe of the plurality of capture probes comprises a spatial barcode and a capture domain;
(b) one or more reagents selected from a buffer, a plurality of dNTPs, a plurality of template switching oligonucleotides (TSOs), a plurality of TSO primers, and a plurality of randomer primers;
(c) one or more enzymes selected from a reverse transcriptase and a polymerase; and
(d) instructions for performing the method of claim 1.
